(19) United States
(12) Patent Application Publication
(10) Pub. No.: US 2025/0296972 A1
(43) Pub. Date: Sep. 25, 2025

(54) KLOTHO DERIVATIVES WITH MODIFIED STRUCTURE

(71) Applicant: ICHILOV TECH LTD., Tel Aviv (IL)

(72) Inventors: Ido WOLF, Tel Aviv (IL); Tami RUBINEK, Or Yehuda (IL); Hagai LIGUMSKY, Tel Aviv (IL); Marana ABBOUD, Tel Aviv (IL)

(73) Assignee: ICHILOV TECH LTD., Tel Aviv (IL)

(21) Appl. No.: 18/863,799

(22) PCT Filed: May 7, 2023
(86) PCT No.: PCT/IL2023/050462

§ 371 Date: Nov. 7, 2024

Related U.S. Application Data
(60) Provisional application No. 63/339,460, filed on May 8, 2022.

(51) Int. Cl. C07K 14/575; A61K 31/7088; A61K 38/22; A61P 35/00; C12N 15/11

(52) U.S. Cl. CPC C07K 14/575; A61K 31/7088; A61K 38/22; A61P 35/00; C12N 15/11

(57) ABSTRACT

Compositions and methods for treating cancer and inhibiting tumor development are provided. More specifically, provided in embodiments of the invention are novel klotho-derived recombinant polypeptides exhibiting tumor suppressor activity, characterized by a distinct structure and improved properties compared to hitherto known klotho polypeptides.

### FIELD OF THE INVENTION

The invention relates to the field of cancer therapy, specifically to compositions and methods for treating cancer and inhibiting tumor development. More specifically, provided in embodiments of the invention are improved klotho-derived polypeptides, exhibiting tumor suppressor activity.

### BACKGROUND OF THE INVENTION

Klotho is a transmembrane protein expressed in a wide spectrum of tissues, that also can be shed to the circulation and act as a hormone. The protein was initially discovered as an ageing-suppressor, and later on identified to play a role in various physiological processes, including, for example, regulation of phosphate and calcium homeostasis.

Membrane-bound human klotho, 1012 amino acids in length, is a single-pass transmembrane protein with an N-terminal signal sequence, an extracellular domain composed of two homologous regions termed KL1 and KL2 (each is approximately 500 amino acids in length), a transmembrane domain and a short intracellular domain. The extracellular domain of klotho contains two recognition sites of metalloproteinases, one between KL2 and the transmembrane domain and another between KL1 and KL2. Cleavage by the metalloproteinases produces soluble klotho proteins that either contain KL1, KL2, or both. It appears that the predominant soluble klotho protein that is shed from the membrane is the one containing both KL1 and KL2. Finally, an alternatively spliced, secreted, form of klotho has been identified, containing the KL1 domain with a tail of additional 15 amino acids at the C-terminus. The secreted form is abundant mainly in the brain.

KL1 and KL2 domains show sequence homology to glycoside hydrolase family 1 enzymes. Crystallographic studies indicated that consistent with this sequence homology, both KL1 domain and KL2 domain contain a triosephosphate isomerase (TIM) barrel fold characteristic of this family of proteins, consisting of an inner eight-stranded parallel β-barrel and eight surrounding α-helices (Chen et al. 2018, Nature, 553, 461-466).

Klotho was found to be a potent tumor suppressor. Its expression is reduced in tumor cells compared to the corresponding normal tissue, and its administration either in vitro or in vivo inhibits growth of cancer cells and alters major cancer-associated signaling pathways.

Ligumsky et al. (2015) Mol Cancer Res., 13(10); 1398-1407, examined the tumor suppressor activities of the KL1 and KL2 domains.

U.S. Pat. Nos. 9,987,326 and 10,555,963, to some of the inventors of the present invention, disclose the use of soluble forms of klotho (e.g., the entire extracellular domain or the KL1 domain), or proteins with at least 80% homology to the amino acid sequence of the soluble forms of klotho, in the treatment of cancer.

WO 2020/039425, to some of the inventors of the present invention, discloses compositions and methods for inhibiting tumor growth in subjects in need thereof, utilizing gene transfer vectors, such as viral vectors, comprising a nucleotide sequence encoding a klotho protein operably linked to at least one regulatory sequence directing its expression.

Despite the potential of using klotho as a tumor suppressor in the treatment of cancer, major technical challenges exist in large-scale production of recombinant klotho proteins, precluding synthesis of klotho or soluble forms thereof such as KL1 in high yields necessary for commercial use. To date, the biopharma industry has been unsuccessful in consistent production of klotho proteins at the appropriate quantity and quality that is required for clinical purposes, and no klotho-based treatment has reached clinical trials. Alternative approaches have been proposed, such as using small molecules that up-regulate expression of klotho, but such approaches have not been successful and may cause toxicity.

Additional compositions and methods for the treatment of cancer are highly desired. In particular, there remains a need for improved therapeutic modalities providing potent anti-tumor activity and amenable with commercial use in the pharmaceutical industry.

### SUMMARY OF THE INVENTION

The invention relates to the field of cancer therapy, specifically to compositions and methods for treating cancer and inhibiting tumor development. More specifically, provided in embodiments of the invention are novel polypeptides derived from klotho KL1 domain, exhibiting tumor suppressor activity, characterized by a distinct structure and improved properties compared to hitherto known klotho polypeptides. Accordingly, advantageous embodiments of the invention provide for improved therapeutic modalities for cancer management, compatible with both large-scale production and clinical use.

The invention is based, in part, on the development of soluble polypeptides derived from the klotho KL1 domain, constructed and evaluated by extensive experimental and in silico analyses. In particular, vectors encoding C-terminal truncated KL1 fragments were generated. These included the whole KL1 region (567 amino acids (aa) long), as well as 365, 340, 320 and 295-aa long polypeptides (prior to cleavage of the signal peptides at positions 1-33), which were further fused at their C′ end to an epitope tag. The anti-cancer activity of the various fragments was evaluated by measuring their ability to inhibit colony formation of pancreatic, breast and colon cancer cells. In addition, their ability to modulate related signaling pathways, in particular the WNT pathway, was also evaluated.

Remarkably, truncated KL1 polypeptides as short as 340 aa in length (prior to cleavage of the signal peptide), successfully inhibited the growth of all tested tumor cell lines, as well as the signaling pathways assayed. Thus, the fragments fully retained all tumor suppressor activities, which were substantially impaired or even completely lost in shorter polypeptides (in which merely 20 additional aa were deleted). The finding that experimentally produced, non-natural fragments lacking substantial portions of the KL1 domain, but retaining at least aa 34-340 thereof, possess anti-tumor activity, is particularly surprising in view of currently available data on the structure-function relations of this protein, as further established by in silico analyses.

In particular, the various activities of klotho were considered to require the triosephosphate isomerase (TIM)-barrel structure, identified to reside in both KL1 and KL2 domains. Surprisingly, a computational analysis revealed that the biologically active 34-340 aa KL1 fragment (herein designated KL340, with or without the signal peptide at positions 1-33) lacks certain amino acids that are considered to be essential for TIM-barrel formation. In other words, KL340 unexpectedly exhibited anti-tumor activity despite a likely perturbation of the TIM-barrel structure, and despite having a predicted conformation that is substantially distinct from the full-length protein (and the KL1 domain) from which it is derived.

Further, by comparison to shorter KL1 fragments in which the observed biological activities were impaired, a newly identified region within the klotho KL1 domain, located between aa 320-340 of full-length klotho, was surprisingly found to be critical for exerting tumor suppressor activities when present in klotho-derived polypeptides.

Accordingly, provided herein are compositions and methods for cancer therapy, employing the use of short KL1-derived polypeptides, which are structurally distinct from hitherto reported klotho polypeptides and soluble fragments thereof (such as KL1 and KL2). The invention in embodiments thereof provides polypeptides exhibiting potent anti-tumor activity, which are amenable for large-scale production in recombinant expression systems.

The production of soluble full-length klotho protein and of its KL1 domain is technically challenging. There are tremendous efforts worldwide to manufacture them under large-scale, GMP conditions, however all attempts (including in bacteria, insect cells and mammalian cells) have failed. Indeed, no klotho-derived treatment is available commercially or has been approved for clinical treatment, despite substantial research investigating this protein.

In some embodiments, the invention provides soluble polypeptides characterized by significantly higher production yields than hitherto reported polypeptides such as KL1. In some embodiments, the invention provides for significantly enhanced production yields in various expression systems including, but not limited to bacterial, insect and eukaryotic cell systems, wherein each possibility represents a separate embodiment of the invention. Without wishing to be bound by a specific theory or mechanism of action, the polypeptides of the invention may provide for improved folding, reduced degradation and/or reduced entrapment in inclusion bodies, wherein each possibility represents a separate embodiment of the invention.

In one aspect, the invention provides a recombinant polypeptide selected from the group consisting of:
a) a polypeptide comprising at least 280 contiguous amino acids of the human klotho polypeptide sequence as set forth in SEQ ID NO: 1 (corresponding to the transcribed human klotho polypeptide as set forth hereinbelow, NM_004795.4), in which the amino acids at positions 341-1012 have been deleted, and at least the amino acids at positions 320-340thereof are retained,
b) a polypeptide of 307-400 amino acids (aa) in length, comprising the amino acids at positions 34-340 of a native human klotho polypeptide precursor sequence, and
c) a polypeptide of at least 280 amino acids in length, having at least 90% sequence identity to a) or b), and exhibiting tumor-suppressive activity.

In one embodiment, the recombinant polypeptide comprises the amino acids at positions 320-340 of SEQ ID NO: 1, as follows: LDFVLGWFAKPVFIDGDYPES (SEQ ID NO: 5). In another embodiment, the recombinant polypeptide comprises, at its C-terminal end, the amino acid sequence of SEQ ID NO: 5, optionally followed by a heterologous sequence or moiety. In another embodiment, the heterologous sequence or moiety is 5-20 amino acids in length.

In another embodiment, the recombinant polypeptide does not form a triosephosphate isomerase (TIM)-barrel structure. In another embodiment, said polypeptide exhibits a tumor suppressor activity that is equivalent to or greater than that exerted by a soluble human klotho polypeptide, wherein each possibility represents a separate embodiment of the invention. In another embodiment, said polypeptide is at least 300 amino acids in length. In another embodiment, said polypeptide is at least 307 amino acids in length.

In another embodiment, the recombinant polypeptide comprises a signal peptide. In some embodiments, the signal peptide is selected from the group consisting of a klotho signal peptide and a heterologous signal peptide. In a particular embodiment said polypeptide comprises the signal peptide at positions 1-33 of SEQ ID NO: 1. In another particular embodiment, said polypeptide is about 340 aa in length. In another embodiment the recombinant polypeptide does not include a signal peptide. In yet another particular embodiment, said polypeptide is about 307 aa in length.

In another embodiment, the recombinant polypeptide consists essentially of the amino acid sequence as set forth in SEQ ID NO: 3 (corresponding to KL340, as set forth below). In another embodiment, the recombinant polypeptide consists essentially of the amino acid sequence as set forth in SEQ ID NO: 3 excluding the signal peptide at positions 1-33 thereof. In another embodiment said polypeptide is encoded by the nucleic acid molecule of SEQ ID NO: 4, as set forth below.

In another embodiment, the recombinant polypeptide further comprises one or more heterologous sequences or moieties. In another embodiment the one or more heterologous sequences or moieties is linked at the C-terminal end. Additionally or alternatively, the one or more heterologous sequences or moieties may be linked at the N-terminal end. In another embodiment the one or more heterologous sequences or moieties is selected from the group consisting of a protein tag, a serum half-life elongating element and a therapeutic agent. Exemplary klotho-derived polypeptides comprising an epitope tag are described in Examples 1 and 3 below. In a particular embodiment the polypeptide further comprises an epitope tag (e.g. an HA tag) linked at the C-terminal end. According to more particular embodiments, said recombinant polypeptide has an amino acid sequence as set forth in SEQ ID NO: 12 or 13, wherein each possibility represents a separate embodiment of the invention.

In another embodiment there is provided a nucleic acid molecule encoding the recombinant polypeptide. In another embodiment the invention provides a recombinant construct, comprising the nucleic acid molecule that is operably linked to one or more transcription regulation sequences. In another embodiment there is provided an expression vector comprising the recombinant construct. In another embodiment there is provided a host cell comprising the expression vector. In another embodiment there is provided an expression system, comprising host cells as disclosed herein contained in a bioreactor.

In another embodiment there is provided a pharmaceutical composition comprising the recombinant polypeptide as disclosed herein. In a particular embodiment the composition comprises a therapeutically effective amount of 0.7-7 mg of said polypeptide. In yet other embodiments, the invention relates to a pharmaceutical composition comprising the construct, vector, or the host cell as disclosed herein.

In another aspect, the invention relates to a pharmaceutical composition comprising a therapeutically effective amount of a recombinant polypeptide as disclosed herein, for use in treating cancer or inhibiting tumor progression in a subject in need thereof. In a further aspect, the invention relates to a pharmaceutical composition comprising a therapeutically effective amount of a construct or vector encoding a recombinant polypeptide as disclosed herein, for use in treating cancer or inhibiting tumor progression in a subject in need thereof.

According to some embodiments, the recombinant polypeptide is selected from the group consisting of: a) a polypeptide comprising at least 280 contiguous amino acids of the human klotho polypeptide sequence as set forth in SEQ ID NO: 1, in which the amino acids at positions 341-1012 have been deleted and at least the amino acids at positions 320-340 thereof are retained, b) a polypeptide of 307-400 aa in length, comprising the amino acids at positions 34-340 of a native human klotho polypeptide precursor sequence, and c) a polypeptide of at least 280 amino acids in length having at least 90% sequence identity to a) or b), and exhibiting tumor-suppressive activity. In another embodiment, the polypeptide comprises, at its C-terminal end, the amino acid sequence of SEQ ID NO: 5, optionally followed by a heterologous sequence or moiety.

In another embodiment, the subject is afflicted with a solid tumor. In some embodiments, the tumor is selected from the group consisting of breast, pancreatic, colorectal, ovarian, cervical and lung tumors, glioblastoma and melanoma. In other specific embodiments, the tumor is selected from the group consisting of pancreatic, breast, ovarian and colon (colorectal) tumors. In other specific embodiments, the tumor is selected from the group consisting of pancreatic, breast and colon tumors. Each possibility represents a separate embodiment of the invention. In another embodiment said composition comprises a therapeutically effective amount of 0.7-7 mg of said polypeptide. In another embodiment said composition is adapted for intravenous administration.

In another aspect, the invention provides a method of treating cancer in a subject in need thereof, comprising administering to the subject, or expressing in cells of the subject, a recombinant polypeptide as disclosed herein, thereby treating cancer in said subject. In some embodiment, the method of treating cancer in a subject comprises administering to the subject a construct or vector encoding a recombinant polypeptide as disclosed herein.

According to some embodiments, the recombinant polypeptide is selected from the group consisting of: a) a polypeptide comprising at least 280 contiguous amino acids of the human klotho polypeptide sequence as set forth in SEQ ID NO: 1, in which the amino acids at positions 341-1012 have been deleted and at least the amino acids at positions 320-340 thereof are retained, b) a polypeptide of 307-400 aa in length, comprising the amino acids at positions 34-340 of a native human klotho polypeptide precursor sequence, and c) a polypeptide of at least 280 amino acids in length having at least 90% sequence identity to a) or b), and exhibiting tumor-suppressive activity. In another embodiment, the polypeptide comprises, at its C-terminal end, the amino acid sequence of SEQ ID NO: 5, optionally followed by a heterologous sequence or moiety. In another embodiment, the subject is afflicted with a solid tumor. In various embodiments, the tumor is selected from the group consisting of breast, pancreatic, colorectal, ovarian, cervical and lung tumors, glioblastoma and melanoma. In other embodiments the tumor is selected from the group consisting of pancreatic, breast and colon tumors. In another embodiment the method comprises administering said polypeptide to said subject at a therapeutically effective amount of 10-100 μg/kg/day. In another embodiment the administration is performed intravenously.

In another aspect there is provided a method of inhibiting tumor progression in a subject in need thereof, comprising administering to the subject a therapeutically effective amount of a polypeptide as disclosed herein, thereby inhibiting tumor progression in said subject. In a further aspect, there is provided a method of inhibiting tumor progression in a subject in need thereof, comprising administering to the subject a therapeutically effective amount of a construct or vector encoding a recombinant polypeptide as disclosed herein, thereby inhibiting tumor progression in said subject.

According to some embodiments, the recombinant polypeptide is selected from the group consisting of: a) a polypeptide comprising at least 280 contiguous amino acids of the human klotho polypeptide sequence as set forth in SEQ ID NO: 1, in which the amino acids at positions 341-1012 have been deleted and at least the amino acids at positions 320-340 thereof are retained, b) a polypeptide of 307-400 aa in length, comprising the amino acids at positions 34-340 of a native human klotho polypeptide precursor sequence, and c) a polypeptide of at least 280 amino acids in length having at least 90% sequence identity to a) or b), and exhibiting tumor-suppressive activity. In another embodiment, the polypeptide comprises, at its C-terminal end, the amino acid sequence of SEQ ID NO: 5, optionally followed by a heterologous sequence or moiety. In another embodiment, the subject is afflicted with a solid tumor. In various embodiments, the tumor is selected from the group consisting of breast, pancreatic, colorectal, ovarian, cervical and lung tumors, glioblastoma and melanoma. In other embodiments the tumor is selected from the group consisting of pancreatic, breast and colon tumors. In another embodiment, the method comprises administering said polypeptide to said subject at a therapeutically effective amount of 10-100 μg/kg/day. In another embodiment the administration is performed intravenously.

In another aspect, there is provided a process of producing a commercial-scale amount of a klotho-derived polypeptide, the process comprising:
a) providing a host cell comprising an expression vector, the vector including a recombinant construct in which a nucleic acid molecule encoding a polypeptide of the invention is operably linked to one or more transcription regulation sequences,
b) culturing the host cell in an expression system comprising a culture vessel, under conditions enabling production of the polypeptide, and
c) collecting the resulting recombinantly-produced polypeptide, to thereby obtain the commercial-scale amount of the klotho-derived polypeptide.

Other objects, features and advantages of the present invention will become clear from the following description and drawings.

### BRIEF DESCRIPTION OF THE DRAWINGS

FIGS. 1A-1B. Construction and expression of truncated klotho variants. (FIG. 1A) A schematic representation of klotho constructs; (FIG. 1B) Exemplary Western blot analysis of MCF-7 cells (breast cancer) transfected with the constructs KL1, KL1-340, KL1-320 or the empty vector pcDNA3 as a control. Western blot was conducted and the klotho truncated proteins were detected using anti-HA antibody. Total protein stain (Ponceau staining) served as a loading control.

FIGS. 2A-2E. Colony formation assay of klotho truncated constructs. MDA-MB-231 (FIG. 2A), MCF-7 (FIG. 2B), HCT116 (FIG. 2C), Panc-1 (FIG. 2D) and MIA PaCa2 (FIG. 2E) cells were seeded in 12 well plates, next day cells were transfected with different KL1 constructs or pcDNA3 as indicated. Forty-eight (48) hours later, cells were trypsinized, transferred to 6-well plates with media containing 750 μg/ml G418 (A, B) or 1300 μg/ml G418 (C) or 900 μg/ml G418 (D, E). After 10 days cells were stained with crystal violet. Colony number was counted using ImageJ program. The experiment was conducted in biological duplicates and was performed three times. The graphs indicate±SD.

FIG. 3. Inhibition of WNT3 expression by klotho truncated constructs. MCF-7 cells were transfected with the klotho constructs or pcDNA3, and with an HA-tagged WNT3 (“WNT3-HA”). After 48 hours cell extracts were loaded on SDS-PAGE gel and immunoblotted with anti-HA to detect WNT3 or with anti β-actin.

FIGS. 4A-4B. Structural analysis of klotho truncated variants. (FIG. 4A) KL1 and the truncated variants KL1-340 and KL1-320 were analyzed by robetta.bakerlab.org website and depicted. Overlay of the three proteins is shown. (FIG. 4B) KL1-340 (red) and amino acids 320-340 (yellow) marked on the reported crystal structure of klotho complexed with FGF23 (teal) and FGFRC1 (magenta). The area of amino acids 320-340 is a putative site suspected to be responsible for klotho tumor suppressor activity.

### DETAILED DESCRIPTION OF THE INVENTION

The invention relates to the field of cancer therapy, specifically to compositions and methods for treating cancer and inhibiting tumor development. More specifically, provided in embodiments of the invention are novel polypeptides derived from klotho KL1 domain, exhibiting tumor suppressor activity, which are characterized by a distinct structure and improved properties compared to hitherto known klotho polypeptides. Accordingly, advantageous embodiments of the invention provide for improved therapeutic modalities for cancer management, compatible with both commercial production and clinical use.

In some embodiments, the invention relates to recombinant polypeptides as disclosed herein, to nucleic acids, constructs and vectors encoding them, to host cells, expression systems and processes for their preparation, and to methods of using same for treating cancer and inhibiting tumor progression. In some embodiments, the recombinant polypeptides are selected from the group consisting of:
a) a polypeptide comprising at least 280 contiguous amino acids of the human klotho polypeptide sequence as set forth in SEQ ID NO: 1 (corresponding to the transcribed human klotho polypeptide as set forth hereinbelow), in which the amino acids at positions 341-1012 have been deleted and at least the amino acids at positions 320-340 thereof are retained,
b) a polypeptide of 307-400 amino acids (aa) in length, comprising the amino acids at positions 34-340 of a native human klotho polypeptide precursor sequence, and
c) a polypeptide of at least 280 amino acids in length having at least 90% sequence identity to a) or b), and exhibiting tumor-suppressive activity.

These and other embodiments are described in further detail below.

The invention in aspects and embodiments thereof relates to non-naturally occurring recombinant and synthetic polypeptides, derived from human klotho. As disclosed herein, the invention in embodiments thereof identifies and defines unique structures and domains, endowing the resulting polypeptides with novel, improved functions and characteristics.

Accordingly, further disclosed herein are novel klotho derivatives that are distinguishable from hitherto known klotho polypeptide by both their structural properties (including primary, secondary and tertiary structures), and functional properties (including, without limitation, with respect to the yield and/or activity upon recombinant production in commercial scale expression systems).

In one aspect, there is provided a klotho-derived polypeptide in which the amino acids C-terminal to position 340 with respect to a human klotho polypeptide sequence have been deleted. In another embodiment the polypeptide is derived from a human klotho polypeptide having an amino acid sequence as set forth in SEQ ID NO: 1 (NM_004795.4 transcribed human klotho polypeptide), in which the amino acids at positions 341-1012 have been deleted. In other embodiments, the klotho-derived polypeptide may be derived from other naturally-occurring allelic variants of human klotho (e.g. as set forth in accession nos. BAA23382.1, KAI4063028.1, KAI2569113.1, BAA24940.1, NP_004786.2, EAX08526.1) that exert a high degree of homology thereto (typically substitutions of one or two amino acids). Advantageously, in the polypeptides of the invention, the amino acids at positions 320-340 with respect to a human klotho polypeptide sequence are retained. In another embodiment the polypeptide comprises the following C-terminal amino acids: LDFVLGWFAKPVFIDGDYPES (SEQ ID NO: 5). In another embodiment, the polypeptide comprises, at its C-terminal end, the amino acid sequence of SEQ ID NO: 5, optionally followed by a heterologous sequence or moiety. In another embodiment the heterologous sequence or moiety is 5-20 amino acids in length.

In another embodiment, the polypeptide is at least 285, 290, 300, 305, 307 or 310 amino acids in length. For example, the invention encompasses in some embodiments certain N-terminal truncations, e.g. of 1-25 amino acids, relative to a klotho-derived polypeptide sequence of the invention as disclosed herein.

In another embodiment, the polypeptide comprises a signal peptide. In various embodiments, the signal peptide is a klotho signal peptide or a heterologous signal peptide. For example, the signal peptide of human klotho resides at positions 1-33 of SEQ ID NO: 1. In another embodiment, the polypeptide does not comprise a signal peptide. In another embodiment, the polypeptide further comprises one or more heterologous sequences, moieties or agents. For example, without limitation, the polypeptide may further comprise a protein tag (e.g. an affinity tag or an epitope tag), a serum half-life elongating sequence (e.g. Fc), or another therapeutic agent (e.g. a toxin or another anti-tumor agent). In one embodiment, the heterologous sequence is at the C′ end. In another embodiment, the heterologous sequence is at the N′end. In another embodiment, the polypeptide does not form a TIM-barrel structure.

In a particular embodiment, the polypeptide has the following amino acid sequence: MPASAPPRRPRPPPPSLSLLLVLLGLGGRRLRAEPGDGAQTWARFSRPPAPEAAGLFQGTFP DGFLWAVGSAAYQTEGGWQQHGKGASIWDTFTHHPLAPPGDSRNASLPLGAPSPLQPATGDV ASDSYNNVERDTEALRELGVTHYRESISWARVLPNGSAGVPNREGLRYYRRLLERLRELGVQ PVVTLYHWDLPQRLQDAYGGWANRALADHERDYAELCFRHEGGQVKYWITIDNPYVVAWHGY ATGRLAPGIRGSPRLGYLVAHNLLLAHAKVWHLYNTSFRPTQGGQVSIALSSHWINPRRMTD HSIKECQKSLDFVLGWFAKPVFIDGDYPES (KL340 polypeptide, SEQ ID NO: 3). In another embodiment said polypeptide consists essentially of SEQ ID NO: 3. In another particular embodiment, said polypeptide lacks the signal peptide at positions 1-33 thereof.

In another embodiment, the polypeptide is encoded by a nucleic acid molecule having the following nucleic acid sequence:

(KL340 cDNA, SEQ ID NO: 4)
ATGCCCGCCAGCGCCCCGCCGCGCCGCCCGCGGCCGCCGCCGCCGTCGCTGTCGCTGCTGCT
GGTGCTGCTGGGCCTGGGCGGCCGCCGCCTGCGTGCGGAGCCGGGCGACGGCGCGCAGACCT
GGGCCCGTTTCTCGCGGCCTCCTGCCCCCGAGGCCGCGGGCCTCTTCCAGGGCACCTTCCCC
GACGGCTTCCTCTGGGCCGTGGGCAGCGCCGCCTACCAGACCGAGGGCGGCTGGCAGCAGCA
CGGCAAGGGTGCGTCCATCTGGGATACGTTCACCCACCACCCCCTGGCACCCCCGGGAGACT
CCCGGAACGCCAGTCTGCCGTTGGGCGCCCCGTCGCCGCTGCAGCCCGCCACCGGGGACGTA
GCCAGCGACAGCTACAACAACGTCTTCCGCGACACGGAGGCGCTGCGCGAGCTCGGGGTCAC
TCACTACCGCTTCTCCATCTCGTGGGCGCGAGTGCTCCCCAATGGCAGCGCGGGCGTCCCCA
ACCGCGAGGGGCTGCGCTACTACCGGCGCCTGCTGGAGCGGCTGCGGGAGCTGGGCGTGCAG
CCCGTGGTCACCCTGTACCACTGGGACCTGCCCCAGCGCCTGCAGGACGCCTACGGCGGCTG
GGCCAACCGCGCCCTGGCCGACCACTTCAGGGATTACGCGGAGCTCTGCTTCCGCCACTTCG
GCGGTCAGGTCAAGTACTGGATCACCATCGACAACCCCTACGTGGTGGCCTGGCACGGCTAC
GCCACCGGGCGCCTGGCCCCCGGCATCCGGGGCAGCCCGCGGCTCGGGTACCTGGTGGCGCA
CAACCTCCTCCTGGCTCATGCCAAAGTCTGGCATCTCTACAATACTTCTTTCCGTCCCACTC
AGGGAGGTCAGGTGTCCATTGCCCTAAGCTCTCACTGGATCAATCCTCGAAGAATGACCGAC
CACAGCATCAAAGAATGTCAAAAATCTCTGGACTTTGTACTAGGTTGGTTTGCCAAACCCGT
ATTTATTGATGGTGACTATCCCGAGAGC.

In another aspect, the invention relates to a recombinant polypeptide of up to 400 aa in length, comprising the amino acids at positions 34-340 of a human klotho polypeptide. In another embodiment, the polypeptide comprises a signal peptide. In various embodiments, the signal peptide is a klotho signal peptide or a heterologous signal peptide. In another embodiment, the polypeptide does not comprise a signal peptide. In some embodiments, the polypeptide is up to 365 aa in length, e.g. 320-355, 330-350 or 335-345 aa in length (e.g. 340 aa together with an N′ klotho signal peptide). In yet other embodiments, the polypeptide is up to about 330 aa in length, e.g. 307-320 or 307-314 aa in length (e.g. without a signal peptide) or in other embodiments 320-340 (e.g. without a signal peptide but including an epitope tag). In another aspect the polypeptide consists essentially of amino acids 34-340 of human klotho (e.g. with respect to SEQ ID NO: 1). In another aspect the polypeptide consists of amino acids 1-340 of human klotho (e.g. of SEQ ID NO: 1). According to particular embodiments, the polypeptide is of the amino acid sequence as set forth in SEQ ID NO: 3 (KL340). In another embodiment, said polypeptide is at least 90% and typically at least 95-98% identical to SEQ ID NO: 3. In another embodiment the polypeptide lacks the signal peptide at positions 1-33 of SEQ ID NO: 3. In another particular embodiment, the polypeptide is encoded by the nucleic acid molecule of SEQ ID NO: 4. In another particular embodiment, said polypeptide is encoded by a nucleic acid sequence as set forth in SEQ ID NO: 9. In yet another embodiment there is provided a recombinant polypeptide encoded a nucleic acid sequence as set forth in SEQ ID NO: 8.

In another embodiment, the polypeptide further comprises one or more heterologous sequences, moieties or agents. For example, without limitation, the polypeptide may further comprise a protein tag (e.g. an affinity tag or an epitope tag), a serum half-life elongating sequence (e.g. Fc), or another therapeutic agent (e.g. a toxin or another anti-tumor agent). In one embodiment, the heterologous sequence is at the C′ end. In another embodiment, the heterologous sequence is at the N′end. In another embodiment, the polypeptide does not form a TIM-barrel structure. Examples 1 and 3 herein describe and exemplify the expression of various sequences and constructs, including additional exemplary polypeptides comprising heterologous sequences. For example, SEQ ID NO: 13 comprises aa 1-340 of human klotho fused at the C′ end to an HA epitope tag.

These recombinant and klotho-derived polypeptides are referred to collectively herein as the polypeptides of the invention.

In another aspect, there is provided a nucleic acid molecule encoding a polypeptide of the invention. In another embodiment there is provided a recombinant construct, comprising the nucleic acid molecule that is operably linked to one or more transcription regulation sequences. In another embodiment there is provided an expression vector comprising the recombinant construct. In another embodiment the vector further comprises a selection marker (e.g. an antibiotic resistance sequence). In another embodiment there is provided a host cell comprising the expression vector. In various embodiments, the host cell is selected from the group consisting of an insect cell, a mammalian cell, a bacterial cell, a yeast cell and a plant cell. In other embodiments, the invention provides a pharmaceutical composition comprising the polypeptide of the invention, or in other embodiments the construct, vector or host cell as disclosed herein. Each possibility represents a separate embodiment of the invention.

In some embodiment, the construct or vector encoding a polypeptide of the invention is a gene therapy vector, for expressing the polypeptide in vivo, in cells of a subject in need of treatment. According to these embodiments, the nucleic acid sequence encoding a polypeptide of the invention is under the control of one or more regulatory sequences which direct expression of the polypeptide in cells of a subject in need of treatment.

The terms “nucleic acid sequence”, “nucleotide sequence” and “polynucleotide” are used herein to refer to polymers of nucleotides, typically deoxyribonucleotides (DNA) and modified forms thereof, in the form of a separate fragment or as a component of a larger construct. A nucleic acid sequence may be a coding sequence, i.e., a sequence that encodes for an end product in the cell, such as a protein/polypeptide. A nucleic acid sequence may also be a regulatory sequence, such as, for example, a promoter.

The term “vector” refers to a man-made composition of matter which comprises a polynucleotide and which is designed for, and can be used to, deliver the polynucleotide to the interior of a target cell.

The term “expression”, as used herein, refers to the cellular process by which a polypeptide is produced based on a nucleic acid sequence. The process includes both transcription and translation.

The term “regulatory sequences” refer to DNA sequences which control the expression (transcription) of coding sequences. For example, regulatory sequences include promoters. The term “promoter” is directed to a regulatory DNA sequence which drives or directs the transcription of another DNA sequence in vivo or in vitro. Usually, the promoter is located in the 5′ region (that is, precedes, located upstream) of the transcribed sequence. Promoters may be derived in their entirety from a native source or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent). Promoters may be ubiquitous or cell/tissue-specific. In most cases the exact boundaries of regulatory sequences have not been completely defined, and in some cases cannot be completely defined, and thus DNA sequences of some variation may have identical promoter activity. The terms “operably linked” and “under the control of” mean that a selected nucleic acid sequence is located with respect to a regulatory sequence (e.g., a promoter) to allow the regulatory sequence to regulate expression of the selected nucleic acid sequence. Promoters suitable for use according to the present invention include ubiquitous promoters that are capable of driving/directing transcription in a constitutive matter in a wide range of cells, particularly in mammalian (preferably human) cells, as well as cell/tissue-specific promoters, such as muscle-specific or liver-specific promoters. Exemplary ubiquitous promoters suitable for use according to the present invention include a Cytomegalovirus (CMV) promoter and Rous sarcoma virus (RSV) promoter. In some particular embodiments, the CMV promoter is CMV-immediate early (CMV-IE) promoter. An exemplary cell/tissue-specific promoter is Desmin promoter, which is muscle-specific.

A “signal peptide” or “signal sequence” refers herein to a short peptide (usually 5-30 amino acids long) typically present at the N-terminus of a newly synthesized polypeptide chain that directs the protein to the secretory pathway in the cell. The signal peptide is typically subsequently removed.

In some embodiments, a vector for expressing a nucleic acid sequence encoding a polypeptide of the invention is a viral vector. Viral vectors for use according to the present invention are recombinant viral vectors. The viral vectors comprise a capsid with a polynucleotide construct packaged therein. The polynucleotide comprises a nucleic acid sequence encoding a polypeptide of the invention. The polynucleotide further comprises at least one regulatory sequence operably linked to the nucleic acid sequence and directing its expression. The at least one regulatory sequence typically comprises a promoter. Additional regulatory sequences include, for example, a polyadenylation signal, a terminator and the like. The polynucleotide in the recombinant viral vector typically further comprises sequences that enable its packaging within the capsid. For example, the polynucleotide typically further comprises inverted terminal repeat (ITR) sequences. In some embodiments, the viral vector is an adeno-associated virus (AAV) vector. According to these embodiments, the viral vector comprises an AAV capsid. In addition, according to these embodiments, the polynucleotide in the capsid comprises AAV ITRs. In some particular embodiments, the viral vector is an AAV9 viral vector. In additional embodiments, the viral vector is an AAV5 viral vector. As used herein, the serotype refers to the capsid, for example, “AAV9 viral vector” indicates an AAV viral vector which contains the capsid of AAV serotype 9.

AAV has a linear single-stranded DNA (ssDNA) genome of approximately 4.7-kilobases (kb), with two 145 nucleotide-long inverted terminal repeats (ITR) at the termini. The virus does not encode a polymerase and therefore relies on cellular polymerases for genome replication. The ITRs flank two viral genes-rep (replication) and cap (capsid), encoding non-structural and structural proteins, respectively. The rep gene, through the use of two promoters and alternative splicing, encodes four regulatory proteins, Rep78, Rep68, Rep52 and Rep40, which are involved in AAV genome replication. The cap gene, through alternative splicing and initiation of translation, gives rise to three capsid proteins, VP1, VP2 and VP3. When AAV infects a human cell alone, its gene expression program is auto-repressed and latency is ensued by preferential integration of the virus genome into a specific region of roughly 2-kb on the long arm of human chromosome 19. This site-specific integration involves the AAV ITRs and Rep proteins. When a latently infected cell is co-infected with a helper virus, such as adenovirus or herpes simplex virus, the AAV gene expression program is activated leading to the AAV Rep-mediated rescue (i.e., excision) of the provirus DNA from the host cell chromosome, followed by replication and packaging of the viral genome. In recombinant AAV vectors (rAAV), the cis-acting viral DNA elements involved in genome amplification and packaging (namely, the ITRs) are typically in linkage with a heterologous sequence of interest (a transgene), whereas the region(s) encoding trans-acting viral factors involved in genome replication and virion assembly (namely, the viral rep and cap genes) are not included and provided in trans. Recombinant AAV vectors may be generated by transfecting producer cells with a plasmid (AAV cis-plasmid) containing a cloned recombinant AAV genome composed of the transgene flanked by the AAV ITRs, and a separate construct expressing in trans the viral rep and cap genes. Helper factors necessary for packaging of the transgene into the capsid are typically provided by transfecting into the producer cells a third plasmid that provides the helper factors. Alternatively, any one or more of the required components (e.g., rep sequences, cap sequences, and/or helper functions) may be provided by a stable producer cell which has been engineered to contain one or more of the required components. The resulting rAAV vector contains a polynucleotide with the transgene flanked by ITRs, packaged inside the capsid. Such rAAV vectors are generally incapable of integrating into the genome of their target cells, as they lack the Rep function. Such rAAV vectors are thought to persist inside the target cells' nucleus as concatemers, providing long-term expression of the transgene. The AAV ITRs, and other selected AAV components described herein, may be readily selected from among any AAV serotype, including, for example, AAV1, AAV2, AAV3, AAV5, AAV6, AAV7, AAV8 and AAV9. Each possibility represents a separate embodiment of the present invention. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like. In some embodiments, the viral vectors of the present invention are AAV vectors that contain a nucleic acid sequence encoding a recombinant polypeptide of the invention and regulatory sequences, flanked by AAV 5′ ITR and AAV 3′ ITR, packaged inside an AAV capsid.

In some embodiments, the methods of the present invention comprise systemically administering a vector (a gene therapy vector, e.g., a viral vector) for expressing a polypeptide of the invention in cells of a subject in need thereof. Systemic administration according the present invention is typically parenteral systemic administration. In some particular embodiments, the methods comprise administering via intramuscular injection. In additional particular embodiments, the methods comprise administering via intravenous injection. In some embodiments, a vector as described herein is administered once. In some embodiments, a single administration of a vector as disclosed herein confers lasting expression of the polypeptide of the invention. For example, a single administration of the vector to a human subject may confer lasting expression for years, e.g., 1-5, 10, 15 years, and possibly more. In other embodiments, the vector is administered at least twice. Recombinant vectors are typically formulated into a pharmaceutical composition suitable for systemic delivery, e.g., intramuscular or intravenous. Such formulation involves the use of pharmaceutically acceptable vehicle(s) or carrier(s), and optionally stabilizing agents, buffers, adjuvants, diluents, etc. For injection, the carrier is typically a liquid. Exemplary physiologically acceptable carriers include sterile water and sterile phosphate buffered saline.

In some embodiments, a range for therapeutically effective amounts of a nucleic acid, nucleic acid construct, vectors or pharmaceutical composition may be from 1×10¹² and 1×10¹³ genome copy (gc)/kg, or from 1×10¹¹ to 1×10¹² gc/kg. It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. For gene therapy vectors, the dosage to be administered may depend to a large extent on the condition and weight of the subject being treated as well as the therapeutic formulation, frequency of treatment and the route of administration.

In some embodiments, the invention relates to systems and processes for recombinant production of a polypeptide of the invention. While the production of naturally-occurring klotho or known fragments thereof (such as those containing the complete KL1 and/or KL2 domains), is challenging, and yields mere microgram amounts of said polypeptides, the invention in advantageous embodiments thereof provides, for the first time, klotho-derived polypeptides amenable for commercial scale or large-scale production. Accordingly, provided in additional embodiments of the invention are systems and processes for recombinant production of a klotho-derived polypeptide under large-scale settings and conditions. In addition, while current commercial-scale production methods of klotho polypeptides provide for non-consistent levels of biologically active polypeptides, the polypeptides in accordance with embodiments of the invention may advantageously be produced at a consistently active form.

In another aspect, there is provided an expression system, comprising a host cell comprising the expression vector as disclosed herein (e.g. that has been transfected, transduced or infected with said vector). In one embodiment, the host cell is immortalized or otherwise compatible with continuous growth. In various embodiments, the host cell is selected from the group consisting of bacterial, plant, yeast, insect and mammalian cells. In another embodiment, the system further comprises a large-scale culture vessel (e.g. a bioreactor) in which host cells as disclosed herein are contained (and may be propagated).

In another aspect, there is provided a process of producing a polypeptide of the invention recombinantly, comprising culturing host cells in an expression system as disclosed herein under conditions enabling production of the polypeptide, and collecting the resulting recombinantly-produced polypeptide. In some embodiments, the expression system is amenable with commercial-scale or large-scale production. In another embodiment, the process further comprises propagating the host cells in a large-scale bioreactor prior to collecting said recombinantly-produced polypeptide.

In another embodiment said process (or system) is characterized in being capable of achieving expression levels of at least 1 mg (or in other embodiments at least 5, 10, 50, 100 or 200 mg) of said polypeptide per liter of culture. In another embodiment, the process (or system) provides yields of at least 1 gram of said polypeptide (or in other embodiments at least 10 or 20 gram).

In some embodiments, particularly suitable expression systems include insect expression systems and mammalian expression systems. Without wishing to be bound by a specific theory or mechanism of action, such expression systems may be advantageous in some embodiments for providing suitably glycosylated polypeptides. For example, without limitation, insect systems including, but not limited to, baculovirus vectors and S2 drosophila host cells, may be used. In addition, mammalian systems utilizing e.g. Chinese hamster ovary (CHO) cells or HEK293 cells, may be used in other embodiments.

To produce a klotho-derived polypeptide in host cells, a nucleic acid molecule encoding the polypeptide may be operably linked to regulatory sequences that control transcriptional expression in an expression vector, and subsequently the expression vector is introduced into the host cells. In addition to transcriptional regulatory sequences, such as promoters, enhancers and terminators, expression vectors may include translational regulatory sequences and a marker gene which is suitable for selection of cells that carry the expression vector. The term “operably linked” refers to a functional linkage of at least two sequences. Operably linked includes a linkage between a promoter and a second sequence, for example a nucleic acid of the present invention, wherein the promoter sequence initiates and mediates transcription of the DNA sequence corresponding to the second sequence.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. Expression vectors may also include nucleotide sequences encoding a secretory sequence that directs the heterologous polypeptide into the secretory pathway of a host cell.

For a mammalian host cell, the transcriptional and translational regulatory signals may be derived from mammalian viral sources, for example, adenovirus, bovine papilloma virus, simian virus, or the like, in which the regulatory signals are associated with a particular gene which has a high level of expression. Suitable transcriptional and translational regulatory sequences also can be obtained from mammalian genes, for example, actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Nat'l Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)). A DNA sequence encoding a klotho-derived polypeptide of the present invention may be operably linked to other/additional genetic elements required for its expression, generally including a transcription promoter and terminator, within an expression vector. The vector typically also contains one or more selectable markers and one or more origins of replication, although those skilled in the art will recognize that within certain systems selectable markers may be provided on separate vectors, and replication of the exogenous DNA may be provided by integration into the host cell genome.

An expression vector may be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

Klotho-derived polypeptides of the present invention may also be produced by cultured mammalian cells using a viral delivery system. Exemplary viruses for this purpose include adenovirus, retroviruses, herpesvirus, vaccinia virus and adeno-associated virus (AAV).

The baculovirus system provides an efficient means to introduce cloned klotho-derived genes into insect cells. Suitable expression vectors are based upon the Autographa californica multiple nuclear polyhedrosis virus (AcMNPV), and contain promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the desired polypeptide into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C-or N-terminus of the expressed polypeptide, for example, a Glu-Glu epitope tag (Grussenmeyer et al., Proc. Nat'l Acad. Sci. 82:7952 (1985)). A transfer vector containing a desired gene is transformed into E. coli, and screened for bacmids which contain an interrupted lacZ gene indicative of recombinant baculovirus. The bacmid DNA containing the recombinant baculovirus genome is then isolated using common techniques.

The recombinant virus or bacmid may be used to transfect host cells, for example insect host cells such as Drosophila Schneider-2 cells. Commercially available serum-free media can be used to grow and to maintain the cells.

Established techniques for producing recombinant proteins in baculovirus systems are provided, for example, by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

A klotho-derived polypeptide expressed in host cells can be isolated by techniques such as affinity chromatography, size exclusion chromatography, ion exchange chromatography, HPLC and the like.

Peptide tags that are useful for isolating heterologous polypeptides expressed by either prokaryotic or eukaryotic cells. Examples include poly-Histidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag, and the FLAG tag (which binds with anti-FLAG antibodies. Affinity tags such as maltose-binding protein or an immunoglobulin domain are also useful for isolating polypeptides of the present invention expressed in host cells. A klotho-derived polypeptide according to the present invention may be produced as a fusion protein with an appropriate peptide/protein tag to facilitate purification. A polynucleotide encoding both components of the fusion protein in the proper reading frame can be generated using known techniques and expressed by the methods described herein.

In another embodiment there is provided a process of producing a polypeptide of the invention in a recombinant manner, the process comprising:
a) providing a host cell comprising an expression vector, the vector including a recombinant construct in which a nucleic acid molecule encoding the polypeptide is operably linked to one or more transcription regulation sequences,
b) culturing the host cell in an expression system comprising a culture vessel, under conditions enabling production of the polypeptide, and
c) collecting the resulting recombinantly-produced polypeptide.

In another embodiment the process further comprises propagating the host cells in the culture vessel prior to collecting said recombinantly-produced polypeptide. In another embodiment, the process is characterized in being capable of achieving expression levels of at least 1 mg of said polypeptide per liter of culture. In another embodiment the process provides yields of at least 1 gr of said polypeptide. In another embodiment the culture vessel is a 300-3000-liter bioreactor, or in other embodiments a 10-300-liter bioreactor. In another embodiment the process yields at least 100 ml of a 1 gr/ml solution.

In other embodiments, the invention relates to methods and processes characterized by improved yield of production compared to known methods and processes for producing klotho polypeptides. In another embodiment, methods and processes of the invention are amenable for producing commercial scale amount (e.g. at least 1 mg per fermentation step) of the klotho-derived polypeptides. In another embodiment, the fraction of misfolded and/or aggregated polypeptide, relative to the total polypeptide produced in a method or process of the invention, is decreased compared to the fraction of misfolded and/or aggregated polypeptide that would be observed under otherwise identical conditions of a known klotho polypeptide, e.g. a soluble human klotho or KL1 domain.

In another aspect, there is provided a process of producing a commercial-scale amount of a klotho-derived polypeptide, the process comprising:
a) providing a host cell comprising an expression vector, the vector including a recombinant construct in which a nucleic acid molecule encoding a polypeptide of the invention is operably linked to one or more transcription regulation sequences,
b) culturing the host cell in an expression system comprising a culture vessel, under conditions enabling production of the polypeptide, and
c) collecting the resulting recombinantly-produced polypeptide,
to thereby obtain the commercial-scale amount of the klotho-derived polypeptide.

In another embodiment the process further comprises propagating the host cells in the culture vessel prior to collecting said recombinantly-produced polypeptide. In another embodiment the amount comprises at least 1 gr of said polypeptide. In various embodiments, said amount comprises 1-100, 10-100, 1-50, 10-50, 50-100 or 25-75 gr. In another embodiment, the process is characterized in being capable of achieving expression levels of at least 1 mg of said polypeptide per liter of culture. In another embodiment the process provides yields of at least 1 gr of said polypeptide. In another embodiment the culture vessel is a 300-3000-liter bioreactor, or in other embodiments a 10-300-liter bioreactor. In another embodiment the process yields at least 100 ml of a 1 gr/ml solution.

In another aspect there is provided a pharmaceutical composition comprising a therapeutically effective amount of a polypeptide produced by the process, which polypeptide exhibits a tumor suppressor activity that is equivalent to or greater than that exerted by a soluble human klotho polypeptide (e.g. a soluble human KL1 polypeptide). In another embodiment the pharmaceutical composition comprises a therapeutically effective amount of 0.7-7 mg of said polypeptide.

In some embodiments, the invention relates to pharmaceutical compositions, comprising a recombinant polypeptide of the invention, and optionally one or more pharmaceutically acceptable carriers, excipients or diluents. In some embodiments, the recombinant polypeptide is selected from the group consisting of:
a) a polypeptide comprising at least 280 contiguous amino acids of the human klotho polypeptide sequence as set forth in SEQ ID NO: 1 (corresponding to the transcribed human klotho polypeptide as set forth hereinbelow), in which the amino acids at positions 341-1012 have been deleted and at least the amino acids at positions 320-340 thereof are retained,
b) a polypeptide of 307-400 amino acids (aa) in length, comprising the amino acids at positions 34-340 of a native human klotho polypeptide precursor sequence, and
c) a polypeptide of at least 280 amino acids in length having at least 90% sequence identity to a) or b), and exhibiting tumor-suppressive activity.

In another embodiment, the polypeptide comprises, at its C-terminal end, the amino acid sequence of SEQ ID NO: 5, optionally followed by a heterologous sequence or moiety. In another embodiment, the polypeptide comprises, consist essentially or consists of the amino acid sequence as set forth in SEQ ID NO: 3 (with or without the signal peptide at positions 1-33). In other embodiments, the active ingredient is a recombinant polypeptide as disclosed herein.

It is to be understood, that the recombinant non-natural polypeptides of the invention contain no more than 400 and typically about 340 contiguous amino acids (aa) or less of a naturally-occurring (native) klotho polypeptide. As such, the polypeptides of the invention exhibit less than 80% homology and typically less than 60% homology to a naturally-occurring klotho polypeptide, or known fragments thereof (such as KL1 and KL2).

As used herein, the term “about”, when referring to the length of a polypeptide according to the present invention, is meant to encompass variations of +/−10 amino acids, preferably variations of +/−5 amino acids compared to the indicated number of amino acids. It is to be understood that the term “about” also encompasses that exact number of amino acids that is indicated.

In addition, polypeptides having at least 90% sequence (e.g., at least 95%, at least 98%, at least 99%) identity to specific polypeptide sequences as disclosed herein are further encompassed in embodiments of the invention, for example those including silent mutations or conservative amino acid substitutions, as known in the art.

In some embodiments, the polypeptide comprises one or more heterologous sequences or moieties at the C-terminal and/or N-terminal end, including, but not limited to, a protein tag, a serum half-life elongating sequence and a therapeutic agent.

In some embodiments, the polypeptide may be fused or conjugated an immunoglobulin or a portion thereof. Other half-life elongating substances include biologically suitable polymers or copolymers, for example, a polyalkylene glycol compound, such as a polyethylene glycol or a polypropylene glycol. Other appropriate polyalkylene glycol compounds include, but are not limited to, charged or neutral polymers of the following types: dextran, polylysine, colominic acids or other carbohydrate-based polymers, polymers of amino acids, and biotin derivatives.

Other examples of the half-life extending moiety, in accordance with the invention, include a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group, an immunoglobulin light chain and heavy chain, an immunoglobulin Fc domain or a portion thereof (see, e.g., U.S. Pat. No. 6,660,843), a CH2 domain of Fc, an albumin (e.g., human serum albumin (HSA)); see, e.g., U.S. Pat. No. 6,926,898 and US 2005/0054051; U.S. Pat. No. 6,887,470), a transthyretin (TTR; see, e.g., US 2003/0195154A1; 2003/0191056 A1), or a thyroxine-binding globulin (TBG).

It should be understood, that the resulting polypeptide or conjugate is selected such that the tumor-suppressive activity is substantially maintained, as described herein. Methods of evaluating tumor-suppressive activity are known in the art. Non-limitative examples of such methods are described in the Examples section below, and include, without limitation, evaluation of the polypeptide's ability to inhibit tumor colony formation, and related signaling pathways. For example, without limitation, polypeptides of the invention typically prevent at least 80% and more typically at least 90% and up to 100% colony formation of breast cancer cells and colorectal cancer cells.

Pharmaceutical compositions may be in any pharmaceutical form suitable for administration to a patient, including but not limited to solutions, suspensions, lyophilized powders for reconstitution with a suitable vehicle or dilution prior to usage, capsules, tablets, sustained-release formulations and the like. The compositions may comprise a therapeutically effective amount of a polypeptide of the present invention, preferably in purified form, and a pharmaceutical excipient. As used herein, “pharmaceutical excipient” includes solvents, dispersion media, coatings, antibacterial and antifungal agents, isotonic and absorption delaying agents etc. and combinations thereof, which are compatible with pharmaceutical administration. Hereinafter, the phrases “therapeutically acceptable carrier” and “pharmaceutically acceptable carrier”, which may be used interchangeably, refer to a carrier or a diluent that does not cause significant irritation to an organism and does not abrogate the biological activity and properties of the administered compound. The compositions may also contain other active compounds providing supplemental, additional, or enhanced therapeutic functions. In another embodiment, the composition consists essentially of the polypeptide of the invention.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. A pharmaceutical composition of the invention is formulated to be compatible with its intended route of administration. Methods to accomplish the administration are known to those of ordinary skill in the art. Examples of suitable excipients and modes for formulating the compositions are described in the latest edition of “Remington's Pharmaceutical Sciences” by E. W. Martin.

Pharmaceutical compositions according to the invention (e.g. containing a recombinant polypeptide as disclosed herein) are typically liquid formulations suitable for injection or infusion. Examples of administration of a pharmaceutical composition include oral ingestion, inhalation, intravenous and continues infusion, intraperitoneal, intramuscular, intracavity, subcutaneous, cutaneous, or transdermal administration. According to certain particular embodiments, the compositions are suitable for intralesional (e.g. intratumoral) administration. In other embodiments, the compositions are particularly suitable for intravenous administration.

For example, saline solutions and aqueous dextrose and glycerol solutions can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Solutions or suspensions used for intravenous administration typically include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin. Where necessary, the composition may also include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Oral compositions include an inert diluent or edible carrier. The composition can be enclosed in gelatin or compressed into tablets. For the purpose of oral administration, the active agent can be incorporated with excipients and placed in tablets, troches, or capsules. Pharmaceutically compatible binding agents or adjuvant materials can be included in the composition. The tablets, troches, and capsules, may optionally contain a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; or a sweetening agent or a flavoring agent.

Solutions or suspensions used for intradermal or subcutaneous application typically include at least one of the following components: a sterile diluent such as water, saline solution, fixed oils, polyethylene glycol, glycerin, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

In some embodiments, the compositions are prepared with carriers to protect the polypeptide against rapid elimination from the body. Biodegradable polymers (e.g., ethylene vinyl acetate, polyanhydrides, polyglycolic acid, collagen, polyorthoesters, and polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811). In other particular embodiments, liposomes containing PEG moieties or glycolipids may advantageously be used to enhance blood plasma retention and/or to reduce liver uptake.

In some embodiments, larger liposomes (e.g. 300 nm or more) are used, which mediate uptake and clearance preferentially by the spleen and having reduced liver clearance. In other embodiments, smaller liposomes (e.g. 40 nm or less) are used, which preferentially mediate liver uptake and clearance. In yet other embodiments, liposomes of 40-300 nm are used, which may have enhanced blood plasma retention. In other embodiments, the liposomes may further contain polymers such as PEG. US 2011160642 discloses pegylated liposomal formulations having reduced accumulation in the liver and spleen. A range of liposomes formulated to evade uptake by the reticuloendothelial system and circulate for longer are described in U.S. Pat. No. 6,284,267.

In addition, the polypeptides of the present invention may be administered with various effector molecules such as heterologous polypeptides, drugs, radionucleotides, or toxins. Thus, in some embodiments, the polypeptide may be administered in combination (concurrent or sequential) with a cancer treatment, e.g. radiation or chemotherapy. In yet other embodiments, the compositions and methods of the invention employ the use of the recombinant polypeptide of the invention as a sole active ingredient.

According to other aspects, the invention is directed to a kit containing a polypeptide of the invention, and instructions to use the polypeptide in the methods of the invention.

According to embodiments of the invention, the pharmaceutical composition comprises the polypeptide of the invention at a therapeutically-effective amount.

An “effective amount” or “therapeutically effective amount” refers to an amount sufficient to exert a beneficial outcome in a method of the invention. More specifically, a therapeutically effective amount means an amount of active ingredients (e.g., a recombinant polypeptide as disclosed herein) effective to prevent, alleviate, or ameliorate symptoms of a disorder (e.g., cancer) or prolong the survival of the subject being treated. Thus, the recombinant polypeptides of the invention may be used in some embodiments in an amount effective to inhibit tumor progression a human subject in need thereof. In a particular embodiment, the therapeutically effective amount is 0.5-10 mg, e.g. 0.7-7 mg. In another particular embodiment, the therapeutically effective amount is 10-100 μg/kg/day.

In another embodiment, there is provided a method of treating cancer in a subject in need thereof, comprising administering to the subject (or expressing in cells of the subject) a polypeptide of the invention, thereby treating cancer in the subject. The term “treating cancer” as used herein should be understood to e.g. encompass treatment resulting in one or more of: a decrease in tumor size; a decrease in rate of tumor growth; stasis of tumor size; a decrease in the number of metastases; a decrease in the number of additional metastasis; a decrease in invasiveness of cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as a decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy. Each possibility represents a separate embodiment of the present invention.

A subject according to the present invention is typically a human subject. In some embodiments, the subject is afflicted with a solid tumor, including, without limitation, a breast, pancreatic, colorectal, ovarian, cervical and lung tumors, glioblastoma and melanoma. In some embodiments, the cancer is selected from the group consisting of pancreatic, breast and colon cancer, wherein each possibility represents a separate embodiment of the invention. In an exemplary embodiment, said polypeptide is administered to said subject at a therapeutically effective amount of 10-100 μg/kg/day, e.g. intravenously.

In another embodiment, there is provided a method of inhibiting tumor progression in a subject in need thereof, comprising administering to the subject (or expressing in cells of the subject) a therapeutically effective amount of a polypeptide of the invention, thereby inhibiting tumor progression in the subject. As used herein, “inhibition” of tumor growth or tumor progression encompasses attenuating, suppressing and even completely arresting the growth of malignant tumors. In some embodiments, the term also encompasses reducing tumor size. “Tumor size” as used herein refers to tumor volume, tumor weight or both. Reduction of tumor size may be complete, namely, destruction of the tumor. In another embodiment the tumor is a solid tumor, including, without limitation, a breast, pancreatic, colorectal, ovarian, cervical and lung tumors, glioblastoma and melanoma. In another embodiment said tumor is selected from the group consisting of pancreatic, breast and colon tumors, wherein each possibility represents a separate embodiment of the invention. In an exemplary embodiment, said polypeptide is administered to said subject at a therapeutically effective amount of 10-100 μg/kg/day, e.g. intravenously.

In other particular embodiments, the dose to be used in the methods of the invention may be 20-80, 30-70 or 40-60 μg/kg/day. Each possibility represents a separate embodiment of the invention.

In various embodiments, a subject to be treated by the compositions and methods of the invention is afflicted with a solid tumor. In some embodiments, the tumor is selected from the group consisting of breast, pancreatic, colorectal, ovarian, cervical and lung tumors, glioblastoma and melanoma. In other specific embodiments, the tumor is selected from the group consisting of pancreatic, breast, ovarian and colon (colorectal) tumors. In other specific embodiments, the tumor is selected from the group consisting of pancreatic, breast and colon tumors. In another embodiment, the tumor is of epithelial origin. In another embodiment the tumor is a carcinoma. In yet another embodiment, said tumor is an adenocarcinoma. Each possibility represents a separate embodiment of the invention.

For example, without limitation, the tumor may be a breast tumor including, but not limited to, an adenocarcinoma or a ductal carcinoma, which may overexpress Her2 and/or the estrogen, progesterone and glucocorticoid receptors. In other examples, an ovarian tumor may be e.g. an ovarian adenocarcinoma or an ovarian serous cystadenocarcinoma. In other exemplary embodiments, a colon cancer may be e.g. a pancreatic carcinoma or ductal adenocarcinoma, which may be metastatic. In other exemplary embodiments, colon cancer may be e.g. colorectal carcinoma, which may contain ras and/or p53 mutations, and/or which may express oncogenes including, but not limited to c-myc, K-ras, H-ras, N-ras, Myb, sis and fos.

The following examples are presented in order to more fully illustrate some embodiments of the invention. They should, in no way be construed, however, as limiting the broad scope of the invention.

### EXAMPLES

### Example 1. Construction of Truncated Klotho-Derived Polypeptides and Evaluation of Their Cancer Inhibition Properties

Plasmids encoding the full-length (wild-type) human KL1 domain and also C-terminal truncated versions of the human KL1 domain were constructed and transfected into breast cancer cell lines, colorectal cell lines and pancreatic cancer cell lines. Colony formation of the cell lines expressing the different constructs was tested in order to evaluate anti-cancer effects of the truncated variants

In particular, the following KL1 polypeptides were constructed (containing the natural signal peptide of human klotho, amino acids 1-33):
“KL1”—full-length human KL1—amino acids 1-567 of human klotho;
“KL1-365”—amino acids 1-365 of human klotho;
“KL1-340”—amino acids 1-340 of human klotho;
“KL1-320”—amino acids 1-320 of human klotho.

### Materials and methods

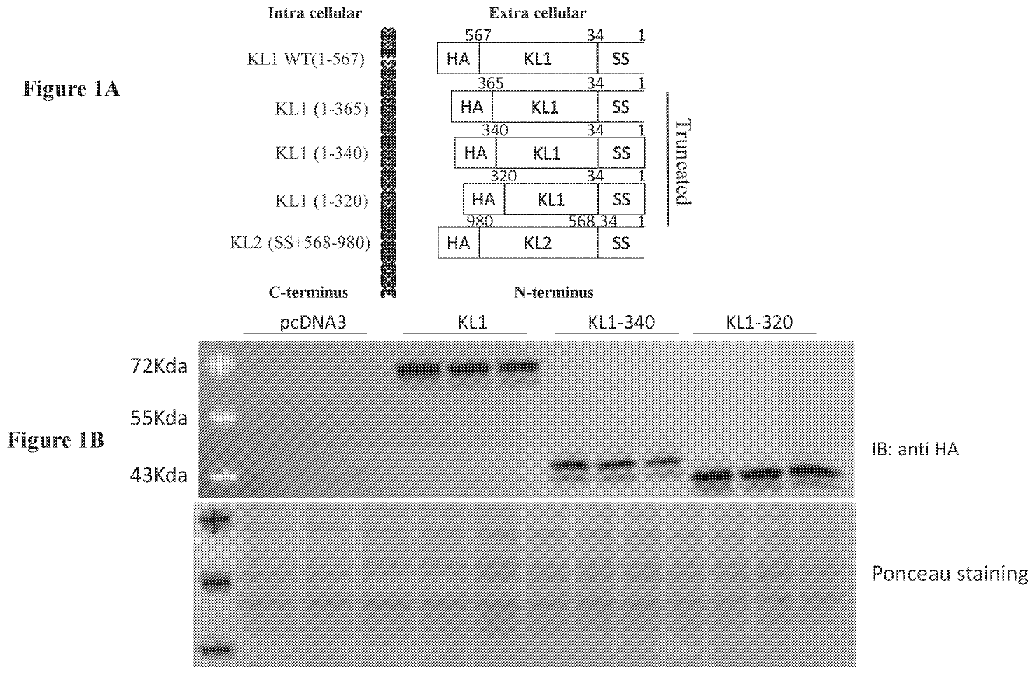

Plasmids: The coding sequence of the entire human klotho protein is set forth herein as SEQ ID NO: 2. The amino acid sequence of human klotho is set forth herein as SEQ ID NO: 1. Nucleic acid sequences encoding KL1, KL1-365, KL1-340 and KL1-320 were cloned between EcoRI and XhoI sites of pcDNA3 by Genscript (Hong-Kong), to obtain nucleic acid constructs encoding each polypeptide tagged with HA at its C-terminus. (FIG. 1A). The KL2 coding sequence, corresponding to nucleotides 1702-2943 of SEQ ID NO: 2, was similarly cloned into pcDNA3, together with the signal sequence (nucleotides 1-99 of SEQ ID NO: 2). The signal sequence was inserted before the KL2 coding sequence, to produce a protein composed of amino acids 1-33 of human klotho (the signal peptide) followed by amino acids 568-980 of human klotho (the KL2 domain) (FIG. 1A).

The constructs in the pcDNA plasmid are expressed under the cytomegalovirus (CMV) promoter. Sequences of the nucleic acid constructs are provided in Example 3 below. These plasmids were also subcloned by excising the EcoRI/XhoI klotho fragments from the HA-tag plasmids into pcDNA3 tagged with Myc. All clones were sequenced following subcloning.

WNT3-HA expression construct, in pcDNA3, was purchased from Upstate Biotechnology (New York, NY, USA).

Cell culture and transfection: MCF-7 (breast cancer), MDA-MB-231 (breast cancer), Panc 1 (pancreatic cancer), MIA PaCa2 (pancreatic cancer) and HCT116 (colorectal cancer) cell lines were purchased from American Type Culture Collection (ATCC) and authenticated with the DNA markers used by ATCC. Cells were cultured at 37° C. in a humidified 5% CO₂ atmosphere in Dulbecco's Modified Eagle's Medium (DMEM) containing 10% fetal calf serum (FCS), 1% penicillin/streptomycin and 1% L-glutamine solution. All transfections used JetPEI® (Polyplus Transfection).

Western blot: The different cell lines were transfected with the klotho constructs or the empty vector pcDNA3 as a control. Cells were washed twice with PBS harvested and lysed for total protein extraction in RIPA buffer (50 mM Tris-HCl, pH 7.4, 150 mM NaCl, 1% NP-40, 0.25% Na-deoxycholate, 1 mM EDTA, 1 mM NaF) together with a protease inhibitor cocktail (Sigma). A total of 50 μg protein extracts were loaded on 10% polyacrylamide gels, separated electrophoretically and blotted onto nitrocellulose membrane (Schleicher & Schuell Bioscience GmbH, Dassel, DE, USA). The membranes were blocked with skim milk 1% and then immunoblotted with the anti-HA (Covance) or anti β-actin (Sigma). Membranes were washed 5 times with TBS-T (0.01M Tris-HCl pH-7.6, 0.15M NaCl, 0.2% Tween 20), followed by incubation with horsereddish peroxidase (HRP) conjugated antibody (Jackson) and detection using ECL reaction.

Colony assay: Cells were seeded in 12-well plates, next day cells were transfected with different KL1 constructs or pcDNA3 as indicated. Forty-eight (48) hours later, cells were trypsinized, transferred to 6-well plates with media containing G418 and grown for 10 days with media exchange twice a week. Following the 10-days incubation, cells were washed twice with PBS and colonies were stained with crystal violet. Colony number was counted using ImageJ program.

### Results

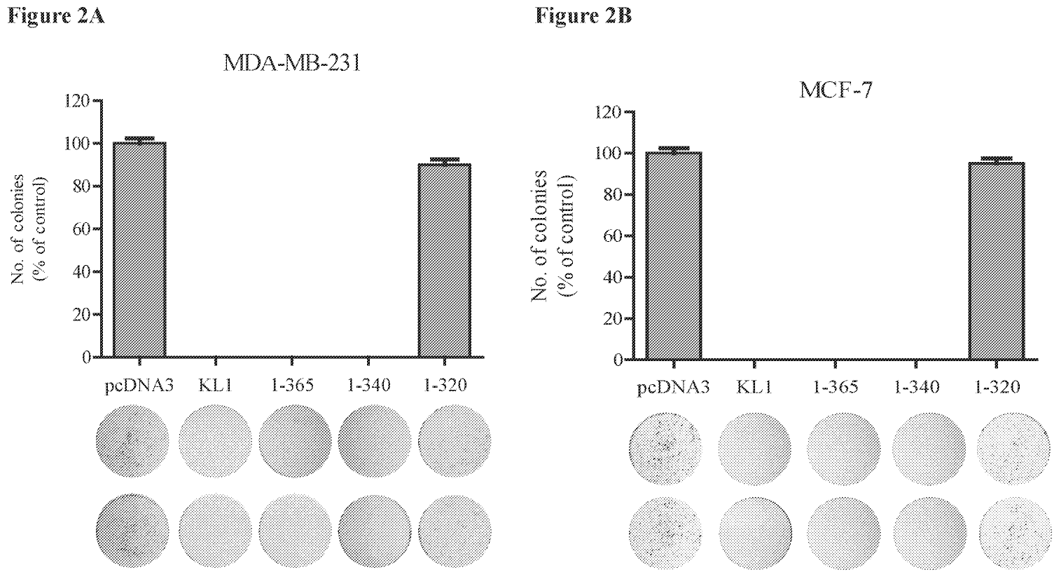

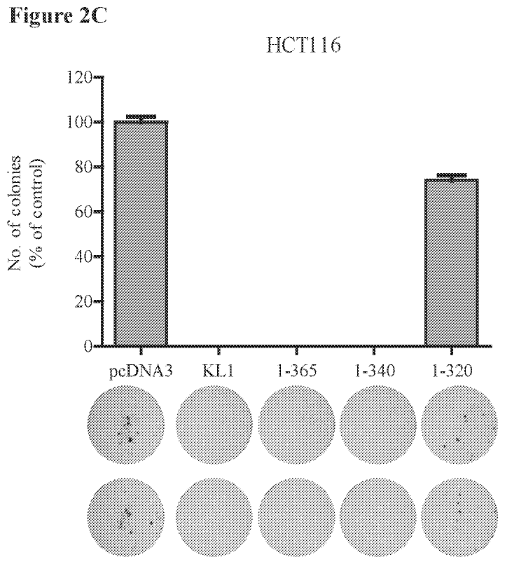

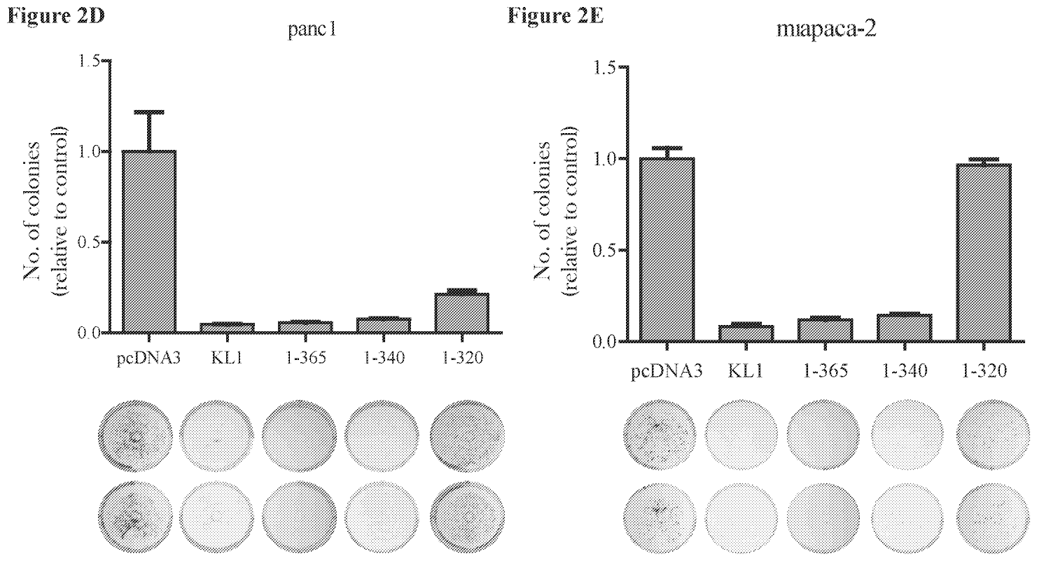

Expression of the different constructs in the cells was validated using Western blot (exemplary results in FIG. 1B). Next, the anti-cancer activity of the different KL1 polypeptides was evaluated using colony formation assay. The plasmids described above were overexpressed in the breast cancer cells MCF-7 and MDA-MB231, colorectal cancer cells HCT116, and in the pancreatic cancer cells PANC-1 and MIA PaCa2. Colony formation was evaluated and the results are summarized in FIGS. 2A-2E. The results unexpectedly showed that KL1-340 and KL1-365 inhibited colony formation to a similar extent as KL1. However, KL1-320 was ineffective, indicating a loss of klotho's anti-cancer activity.

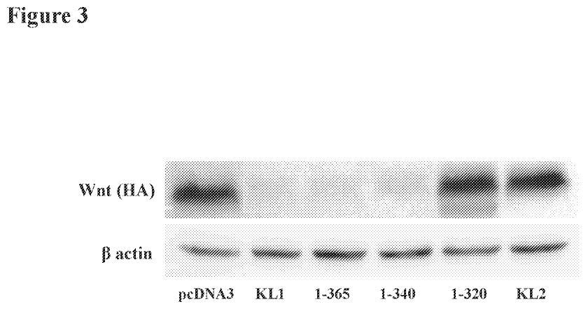

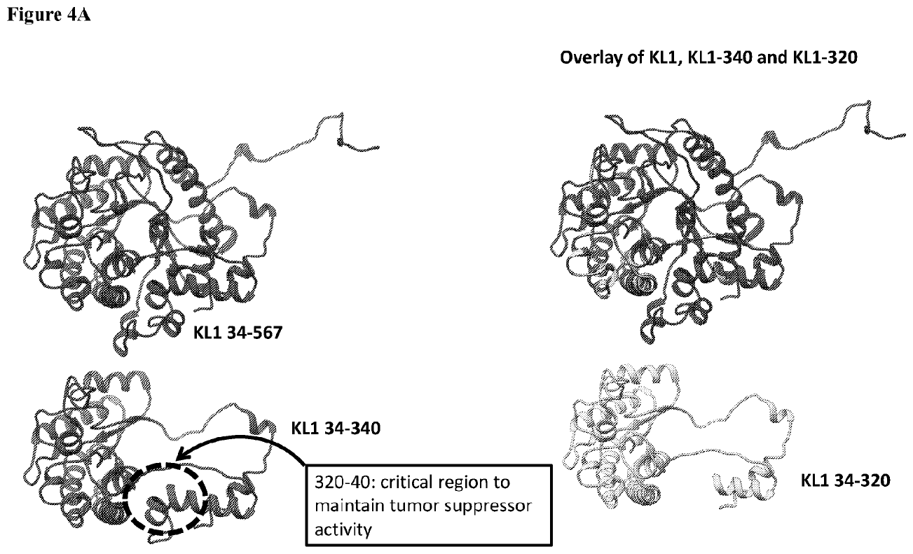

The anti-cancer activity of the KL1 polypeptides was also evaluated by their ability to inhibit relevant signaling pathways, particularly the WNT signaling pathway. To this end, MCF-7 cells were transfected with an HA-tagged WNT3 (“WNT3-HA”) and with either KL1, KL1-365, KL1-340, KL1-320 or KL2. WNT3-HA expression was assessed using Western blot. The results are summarized in FIG. 3. The results showed that while KL1, KL1-365 and KL1-340 reduced WNT3-HA expression, KL1-320 and KL2 did not affect WNT3-HA expression.

Thus, the results demonstrate that truncated klotho-derived polypeptides as short as 340 aa in length (prior to cleavage of the signal peptide), successfully inhibited the growth of all tested tumor cell lines, as well as the signaling pathways assayed.

The experiments are repeated in additional cell lines, including: breast cancer cell lines (in addition to MCF7-SKBR3, MDAMB231, BT474, and T47D cells), ovarian cancer cell lines (SKOV3 and OVCAR3 cells), pancreatic cancer cell lines (PANCI, MiaPaCa2, and Colo357), and colorectal cancer cell lines (HCT116, SW480, HT29).

For example, MCF-7 is a human breast cancer cell line (adenocarcinoma) with estrogen, progesterone and glucocorticoid receptors. SK-BR-3 is a human breast cancer cell line (adenocarcinoma) that overexpresses the Her2 (Neu/ErbB-2) gene product. MDA-MB-231 are human adenocarcinoma cells. BT474 are ductal carcinoma cells overexpressing Her2. T47D is a ductal carcinoma line characterized in that their progesterone receptors are not regulated by estradiol.

In other examples, SKOV3 is an ovarian serous cystadenocarcinoma line and OVCAR3is an ovarian adenocarcinoma cell line. PANC-1 is a human ductal cell pancreatic carcinoma cell line, MiaPaCa2 is a ductal adenocarcinoma line, and Colo357 is a cell line derived from a metastasis of a pancreatic adenocarcinoma.

In other examples, HCT116 is a human colorectal carcinoma cell line, having a mutation in codon 13 of the ras proto-oncogene. SW480 is a cell line established from a primary adenocarcinoma of the colon. HT29 is a human colorectal adenocarcinoma cell line containing a p53 mutation resulting in overexpression and positive for expression of c-myc, K-ras, H-ras, N-ras, Myb, sis and fos oncogenes.

### Example 2. Structural Analysis of Klotho-Derived Polypeptides

Data derived from crystallography (Chen et al., 2018, Nature, 553: 461-466) and computer models (Ligumsky et al., 2015, Mol. Cancer Res., 13: 1398-1407; Wright et al., 2019, FASEB J. Off. Publ. Fed. Am. Soc. Exp. Biol. 33: 9182-9193) showed that the KL1 and KL2 domains of klotho have a triosephosphate isomerase (TIM)-barrel structure, which resembles the TIM-barrel structure of the beta-glucosidase family, particularly that of klotho-related protein (KLRP). To evaluate the effect of the truncation on the protein structure, KL1, KL1-340 and KL1-320 were computer-modelled and compared as described below.

Computer modelling was carried out using robetta.bakerlab.org website, and also using as a template structure for molecular modeling the human klotho that was recently resolved in a 1:1:1 FGF23-FGFR1c-aKlotho ternary complex (PDB ID entry 5W21) with 3 Å resolution (Chen et al., supra).

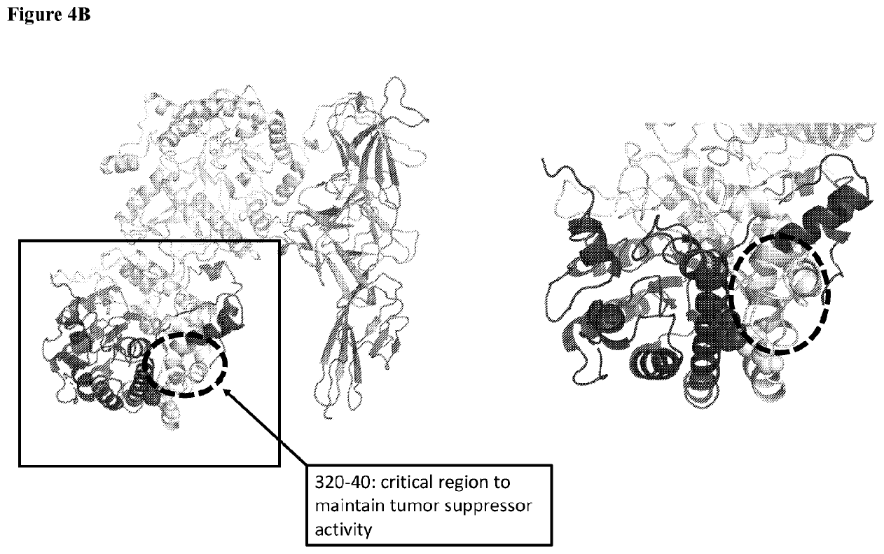

The results, shown in FIGS. 4A-4B, indicate that KL1-340 lacks amino acids that are considered to be essential for TIM-barrel formation, and is not expected to fold into a TIM-barrel, as several of the helices and loops crucial for this fold are missing. As shown in Example 1, KL1-340 retains the anti-cancer activity of klotho. Thus, it is unexpectedly disclosed that the anti-cancer activity of KL1-340 is retained despite a likely perturbation of the TIM-barrel structure.

A further inspection of the computerized model of the biologically active KL1-340 polypeptide in comparison to KL1-320 that was found to be inactive, showed that amino acids 320-340 are part of an alpha-helix. Omitting these 20 amino acids shortens the helix, which may disturb the stability of the protein (FIG. 4A-4B). It was concluded that amino acids 320-340 of the klotho sequence, which are likely to adopt an alpha helix structure, are required for the anti-cancer activity of KL1. Thus, a new region was identified within the KL1 domain of klotho, which is critical for maintaining tumor suppressor activity.

### Example 3. Construct Sequences

All constructs are based on NCBI accession number NM_004795.4.

### Human KL1 Cloned in pcDNA3 with a C-Terminal HA Tag: (SEQ ID NO: 6)

Restriction sites of EcoRI and XhoI are marked in Italics.

The ATG start codon and the last codon of KL1 are underlined and marked in boldface. The signal sequence is underlined.

The sequence encoding the HA tag is marked in boldface. The TGA stop codon (following the HA tag) is underlined and marked in Italics.

Sequence:

GGATCCGAATTCATGCCCGCCAGCGCCCCGCCGCGCCGCCCGCGGCCGCCGCCGCCGTCGCT
GTCGCTGCTGCTGGTGCTGCTGGGCCTGGGCGGCCGCCGCCTGCGTGCGGAGCCGGGCGACG
GCGCGCAGACCTGGGCCCGTTTCTCGCGGCCTCCTGCCCCCGAGGCCGCGGGCCTCTTCCAG
GGCACCTTCCCCGACGGCTTCCTCTGGGCCGTGGGCAGCGCCGCCTACCAGACCGAGGGCGG
CTGGCAGCAGCACGGCAAGGGTGCGTCCATCTGGGATACGTTCACCCACCACCCCCTGGCAC
CCCCGGGAGACTCCCGGAACGCCAGTCTGCCGTTGGGCGCCCCGTCGCCGCTGCAGCCCGCC
ACCGGGGACGTAGCCAGCGACAGCTACAACAACGTCTTCCGCGACACGGAGGCGCTGCGCGA
GCTCGGGGTCACTCACTACCGCTTCTCCATCTCGTGGGCGCGAGTGCTCCCCAATGGCAGCG
CGGGCGTCCCCAACCGCGAGGGGCTGCGCTACTACCGGCGCCTGCTGGAGCGGCTGCGGGAG
CTGGGCGTGCAGCCCGTGGTCACCCTGTACCACTGGGACCTGCCCCAGCGCCTGCAGGACGC
CTACGGCGGCTGGGCCAACCGCGCCCTGGCCGACCACTTCAGGGATTACGCGGAGCTCTGCT
TCCGCCACTTCGGCGGTCAGGTCAAGTACTGGATCACCATCGACAACCCCTACGTGGTGGCC
TGGCACGGCTACGCCACCGGGCGCCTGGCCCCCGGCATCCGGGGCAGCCCGCGGCTCGGGTA
CCTGGTGGCGCACAACCTCCTCCTGGCTCATGCCAAAGTCTGGCATCTCTACAATACTTCTT
TCCGTCCCACTCAGGGAGGTCAGGTGTCCATTGCCCTAAGCTCTCACTGGATCAATCCTCGA
AGAATGACCGACCACAGCATCAAAGAATGTCAAAAATCTCTGGACTTTGTACTAGGTTGGTT
TGCCAAACCCGTATTTATTGATGGTGACTATCCCGAGAGCATGAAGAATAACCTTTCATCTA
TTCTGCCTGATTTTACTGAATCTGAGAAAAAGTTCATCAAAGGAACTGCTGACTTTTTTGCT
CTTTGCTTTGGACCCACCTTGAGTTTTCAACTTTTGGACCCTCACATGAAGTTCCGCCAATT
GGAATCTCCCAACCTGAGGCAACTGCTTTCCTGGATTGACCTTGAATTTAACCATCCTCAAA
TATTTATTGTGGAAAATGGCTGGTTTGTCTCAGGGACCACCAAGAGAGATGATGCCAAATAT
ATGTATTACCTCAAAAAGTTCATCATGGAAACCTTAAAAGCCATCAAGCTGGATGGGGTGGA
TGTCATCGGGTATACCGCATGGTCCCTCATGGATGGTTTCGAGTGGCACAGAGGTTACAGCA
TCAGGCGTGGACTCTTCTATGTTGACTTTCTAAGCCAGGACAAGATGTTGTTGCCAAAGTCT
TCAGCCTTGTTCTACCAAAAGCTGATAGAGAAAAATGGCTTCCCTCCTTTACCTGAAAATCA
GCCCCTAGAAGGGACATTTCCCTGTGACTTTGCTTGGGGAGTTGTTGACAACTACATTCAAG
TAGATACCACTCTGTCTCAGTTTACCGACCTGAATGTTTACCTGTGGGATGTCCACCACAGT
AAAAGGCTTATTAAAGTGGATGGGGTTGTGACCAAGAAGCTCGAGTCTAGAGGGCCCTACCC
ATACGATGTTCCAGATTACGCTTGATA

### Human KL1 with a C-Terminal HA Tag, Amino Acid Sequence: (SEQ ID NO: 7)

The last amino acid of KL1 is underlined and marked in boldface. The HA tag is marked in boldface

Sequence:

MPASAPPRRPRPPPPSLSLLLVLLGLGGRRLRAEPGDGAQTWARFSRPPAPEAAGLFQGTFP
DGFLWAVGSAAYQTEGGWQQHGKGASIWDTFTHHPLAPPGDSRNASLPLGAPSPLQPATGDV
ASDSYNNVFRDTEALRELGVTHYRFSISWARVLPNGSAGVPNREGLRYYRRLLERLRELGVQ
PVVTLYHWDLPQRLQDAYGGWANRALADHFRDYAELCFRHFGGQVKYWITIDNPYVVAWHGY
ATGRLAPGIRGSPRLGYLVAHNLLLAHAKVWHLYNTSFRPTQGGQVSIALSSHWINPRRMTD
HSIKECQKSLDFVLGWFAKPVFIDGDYPESMKNNLSSILPDFTESEKKFIKGTADFFALCFG
PTLSFQLLDPHMKFRQLESPNLRQLLSWIDLEFNHPQIFIVENGWFVSGTTKRDDAKYMYYL
KKFIMETLKAIKLDGVDVIGYTAWSLMDGFEWHRGYSIRRGLFYVDFLSQDKMLLPKSSALF
YQKLIEKNGFPPLPENQPLEGTFPCDFAWGVVDNYIQVDTTLSQFTDLNVYLWDVHHSKRLI
KVDGVVTKKLESRGPYPYDVPDYA-

### KL1-365 cloned in pcDNA3 with a C-Terminal HA Tag: (SEQ ID NO: 8)

Restriction sites of EcoRI and XhoI are marked in Italics.

The ATG start codon and the last codon of KL1 are underlined and marked in boldface. The signal sequence is underlined.

The sequence encoding the HA tag is marked in boldface. The TGA stop codon (following the HA tag) is underlined and marked in Italics.

Sequence:

GGATCCGAATTCATGCCCGCCAGCGCCCCGCCGCGCCGCCCGCGGCCGCCGCCGCCGTCGCT
GTCGCTGCTGCTGGTGCTGCTGGGCCTGGGCGGCCGCCGCCTGCGTGCGGAGCCGGGCGACG
GCGCGCAGACCTGGGCCCGTTTCTCGCGGCCTCCTGCCCCCGAGGCCGCGGGCCTCTTCCAG
GGCACCTTCCCCGACGGCTTCCTCTGGGCCGTGGGCAGCGCCGCCTACCAGACCGAGGGCGG
CTGGCAGCAGCACGGCAAGGGTGCGTCCATCTGGGATACGTTCACCCACCACCCCCTGGCAC
CCCCGGGAGACTCCCGGAACGCCAGTCTGCCGTTGGGCGCCCCGTCGCCGCTGCAGCCCGCC
ACCGGGGACGTAGCCAGCGACAGCTACAACAACGTCTTCCGCGACACGGAGGCGCTGCGCGA
GCTCGGGGTCACTCACTACCGCTTCTCCATCTCGTGGGCGCGAGTGCTCCCCAATGGCAGCG
CGGGCGTCCCCAACCGCGAGGGGCTGCGCTACTACCGGCGCCTGCTGGAGCGGCTGCGGGAG
CTGGGCGTGCAGCCCGTGGTCACCCTGTACCACTGGGACCTGCCCCAGCGCCTGCAGGACGC
CTACGGCGGCTGGGCCAACCGCGCCCTGGCCGACCACTTCAGGGATTACGCGGAGCTCTGCT
TCCGCCACTTCGGCGGTCAGGTCAAGTACTGGATCACCATCGACAACCCCTACGTGGTGGCC
TGGCACGGCTACGCCACCGGGCGCCTGGCCCCCGGCATCCGGGGCAGCCCGCGGCTCGGGTA
CCTGGTGGCGCACAACCTCCTCCTGGCTCATGCCAAAGTCTGGCATCTCTACAATACTTCTT
TCCGTCCCACTCAGGGAGGTCAGGTGTCCATTGCCCTAAGCTCTCACTGGATCAATCCTCGA
AGAATGACCGACCACAGCATCAAAGAATGTCAAAAATCTCTGGACTTTGTACTAGGTTGGTT
TGCCAAACCCGTATTTATTGATGGTGACTATCCCGAGAGCATGAAGAATAACCTTTCATCTA
TTCTGCCTGATTTTACTGAATCTGAGAAAAAGTTCATCAAAGGAACTGCTGACCTCGAGTCT
AGAGGGCCCTACCCATACGATGTTCCAGATTACGCTTGATA

### KL1-365 with a C-Terminal HA Tag, Amino Acid Sequence: (SEQ ID NO: 12)

The last amino acid of KL1 is underlined and marked in boldface. The HA tag is marked in boldface

Sequence:

MPASAPPRRPRPPPPSLSLLLVLLGLGGRRLRAEPGDGAQTWARFSRPPAPEAAGLFQGTFP
DGFLWAVGSAAYQTEGGWQQHGKGASIWDTFTHHPLAPPGDSRNASLPLGAPSPLQPATGDV
ASDSYNNVFRDTEALRELGVTHYRFSISWARVLPNGSAGVPNREGLRYYRRLLERLRELGVQ
PVVTLYHWDLPQRLQDAYGGWANRALADHFRDYAELCFRHFGGQVKYWITIDNPYVVAWHGY
ATGRLAPGIRGSPRLGYLVAHNLLLAHAKVWHLYNTSFRPTQGGQVSIALSSHWINPRRMTD
HSIKECQKSLDFVLGWFAKPVFIDGDYPESMKNNLSSILPDFTESEKKFIKGTADLESRGPY
PYDVPDYA-

### KL1-340 Cloned in pcDNA3 with a C-Terminal HA Tag: (SEQ ID NO: 9)

Restriction sites of EcoRI and XhoI are marked in Italics.

The ATG start codon and the last codon of KL1 are underlined and marked in boldface. The signal sequence is underlined.

The sequence encoding the HA tag is marked in boldface. The TGA stop codon (following the HA tag) is underlined and marked in Italics.

Sequence:

GAATTCATGCCCGCCAGCGCCCCGCCGCGCCGCCCGCGGCCGCCGCCGCCGTCGCTGTCGCT
GCTGCTGGTGCTGCTGGGCCTGGGCGGCCGCCGCCTGCGTGCGGAGCCGGGCGACGGCGCGC
AGACCTGGGCCCGTTTCTCGCGGCCTCCTGCCCCCGAGGCCGCGGGCCTCTTCCAGGGCACC
TTCCCCGACGGCTTCCTCTGGGCCGTGGGCAGCGCCGCCTACCAGACCGAGGGCGGCTGGCA
GCAGCACGGCAAGGGTGCGTCCATCTGGGATACGTTCACCCACCACCCCCTGGCACCCCCGG
GAGACTCCCGGAACGCCAGTCTGCCGTTGGGCGCCCCGTCGCCGCTGCAGCCCGCCACCGGG
GACGTAGCCAGCGACAGCTACAACAACGTCTTCCGCGACACGGAGGCGCTGCGCGAGCTCGG
GGTCACTCACTACCGCTTCTCCATCTCGTGGGCGCGAGTGCTCCCCAATGGCAGCGCGGGCG
TCCCCAACCGCGAGGGGCTGCGCTACTACCGGCGCCTGCTGGAGCGGCTGCGGGAGCTGGGC
GTGCAGCCCGTGGTCACCCTGTACCACTGGGACCTGCCCCAGCGCCTGCAGGACGCCTACGG
CGGCTGGGCCAACCGCGCCCTGGCCGACCACTTCAGGGATTACGCGGAGCTCTGCTTCCGCC
ACTTCGGCGGTCAGGTCAAGTACTGGATCACCATCGACAACCCCTACGTGGTGGCCTGGCAC
GGCTACGCCACCGGGCGCCTGGCCCCCGGCATCCGGGGCAGCCCGCGGCTCGGGTACCTGGT
GGCGCACAACCTCCTCCTGGCTCATGCCAAAGTCTGGCATCTCTACAATACTTCTTTCCGTC
CCACTCAGGGAGGTCAGGTGTCCATTGCCCTAAGCTCTCACTGGATCAATCCTCGAAGAATG
ACCGACCACAGCATCAAAGAATGTCAAAAATCTCTGGACTTTGTACTAGGTTGGTTTGCCAA
ACCCGTATTTATTGATGGTGACTATCCCGAGAGCCTCGAGTCTAGAGGGCCCTACCCATACG
ATGTTCCAGATTACGCTTGATA

### KL1-340 with a C-Terminal HA Tag, Amino Acid Sequence: (SEQ ID NO: 13)

The last amino acid of KL1 is underlined and marked in boldface. The HA tag is marked in boldface

Sequence:

MPASAPPRRPRPPPPSLSLLLVLLGLGGRRLRAEPGDGAQTWARFSRPPAPEAAGLFQGTFP
DGFLWAVGSAAYQTEGGWQQHGKGASIWDTFTHHPLAPPGDSRNASLPLGAPSPLQPATGDV
ASDSYNNVFRDTEALRELGVTHYRFSISWARVLPNGSAGVPNREGLRYYRRLLERLRELGVQ
PVVTLYHWDLPQRLQDAYGGWANRALADHFRDYAELCFRHFGGQVKYWITIDNPYVVAWHGY
ATGRLAPGIRGSPRLGYLVAHNLLLAHAKVWHLYNTSFRPTQGGQVSIALSSHWINPRRMTD
HSIKECQKSLDFVLGWFAKPVFIDGDYPESLESRGPYPYDVPDYA-

### KL1-320 Cloned in pcDNA3 with a C-Terminal HA Tag: (SEQ ID NO: 10)

Restriction sites of EcoRI and XhoI are marked in Italics.

The ATG start codon and the last codon of KL1 are underlined and marked in boldface. The signal sequence is underlined.

The sequence encoding the HA tag is marked in boldface. The TGA stop codon (following the HA tag) is underlined and marked in Italics.

Sequence:

GAATTCATGCCCGCCAGCGCCCCGCCGCGCCGCCCGCGGCCGCCGCCGCCGTCGCTGTCGCT
GCTGCTGGTGCTGCTGGGCCTGGGCGGCCGCCGCCTGCGTGCGGAGCCGGGCGACGGCGCGC
AGACCTGGGCCCGTTTCTCGCGGCCTCCTGCCCCCGAGGCCGCGGGCCTCTTCCAGGGCACC
TTCCCCGACGGCTTCCTCTGGGCCGTGGGCAGCGCCGCCTACCAGACCGAGGGCGGCTGGCA
GCAGCACGGCAAGGGTGCGTCCATCTGGGATACGTTCACCCACCACCCCCTGGCACCCCCGG
GAGACTCCCGGAACGCCAGTCTGCCGTTGGGCGCCCCGTCGCCGCTGCAGCCCGCCACCGGG
GACGTAGCCAGCGACAGCTACAACAACGTCTTCCGCGACACGGAGGCGCTGCGCGAGCTCGG
GGTCACTCACTACCGCTTCTCCATCTCGTGGGCGCGAGTGCTCCCCAATGGCAGCGCGGGCG
TCCCCAACCGCGAGGGGCTGCGCTACTACCGGCGCCTGCTGGAGCGGCTGCGGGAGCTGGGC
GTGCAGCCCGTGGTCACCCTGTACCACTGGGACCTGCCCCAGCGCCTGCAGGACGCCTACGG
CGGCTGGGCCAACCGCGCCCTGGCCGACCACTTCAGGGATTACGCGGAGCTCTGCTTCCGCC
ACTTCGGCGGTCAGGTCAAGTACTGGATCACCATCGACAACCCCTACGTGGTGGCCTGGCAC
GGCTACGCCACCGGGCGCCTGGCCCCCGGCATCCGGGGCAGCCCGCGGCTCGGGTACCTGGT
GGCGCACAACCTCCTCCTGGCTCATGCCAAAGTCTGGCATCTCTACAATACTTCTTTCCGTC
CCACTCAGGGAGGTCAGGTGTCCATTGCCCTAAGCTCTCACTGGATCAATCCTCGAAGAATG
ACCGACCACAGCATCAAAGAATGTCAAAAATCTCTGCTCGAGTCTAGAGGGCCCTACCCATA
CGATGTTCCAGATTACGCTTGATA.

### KL1-320 with a C-Terminal HA Tag, Amino Acid Sequence: (SEQ ID NO: 14)

The last amino acid of KL1 is underlined and marked in boldface. The HA tag is marked in boldface

Sequence:

MPASAPPRRPRPPPPSLSLLLVLLGLGGRRLRAEPGDGAQTWARFSRPPAPEAAGLFQGTFP
DGFLWAVGSAAYQTEGGWQQHGKGASIWDTFTHHPLAPPGDSRNASLPLGAPSPLQPATGDV
ASDSYNNVFRDTEALRELGVTHYRFSISWARVLPNGSAGVPNREGLRYYRRLLERLRELGVQ
PVVTLYHWDLPQRLQDAYGGWANRALADHFRDYAELCFRHEGGQVKYWITIDNPYVVAWHGY
ATGRLAPGIRGSPRLGYLVAHNLLLAHAKVWHLYNTSFRPTQGGQVSIALSSHWINPRRMTD
HSIKECQKSLLESRGPYPYDVPDYA-

### Human KL2 Cloned in pcDNA3 with a Signal Peptide and a C-Terminal HA Tag: (SEQ ID NO: 11)

Restriction sites of EcoRI and XhoI are marked in Italics.

The ATG start codon and the last codon of KL2 are underlined and marked in boldface. The signal sequence is underlined.

The sequence encoding the HA tag is marked in boldface. The TGA stop codon (following the HA tag) is underlined and marked in Italics.

Sequence:

GAATTCATGCCCGCCAGCGCCCCGCCGCGCCGCCCGCGGCCGCCGCCGCCGTCGCTGTCGCT
GCTGCTGGTGCTGCTGGGCCTGGGCGGCCGCCGCCTGCGTGCGAGGAAATCCTACTGTGTTG
ACTTTGCTGCCATCCAGCCCCAGATCGCTTTACTCCAGGAAATGCACGTTACACATTTTCGC
TTCTCCCTGGACTGGGCCCTGATTCTCCCTCTGGGTAACCAGTCCCAGGTGAACCACACCAT
CCTGCAGTACTATCGCTGCATGGCCAGCGAGCTTGTCCGTGTCAACATCACCCCAGTGGTGG
CCCTGTGGCAGCCTATGGCCCCGAACCAAGGACTGCCGCGCCTCCTGGCCAGGCAGGGCGCC
TGGGAGAACCCCTACACTGCCCTGGCCTTTGCAGAGTATGCCCGACTGTGCTTTCAAGAGCT
CGGCCATCACGTCAAGCTTTGGATAACGATGAATGAGCCGTATACAAGGAATATGACATACA
GTGCTGGCCACAACCTTCTGAAGGCCCATGCCCTGGCTTGGCATGTGTACAATGAAAAGTTT
AGGCATGCTCAGAATGGGAAAATATCCATAGCCTTGCAGGCTGATTGGATAGAACCTGCCTG
CCCTTTCTCCCAAAAGGACAAAGAGGTGGCCGAGAGAGTTTTGGAATTTGACATTGGCTGGC
TGGCTGAGCCCATTTTCGGCTCTGGAGATTATCCATGGGTGATGAGGGACTGGCTGAACCAA
AGAAACAATTTTCTTCTTCCTTATTTCACTGAAGATGAAAAAAAGCTAATCCAGGGTACCTT
TGACTTTTTGGCTTTAAGCCATTATACCACCATCCTTGTAGACTCAGAAAAAGAAGATCCAA
TAAAATACAATGATTACCTAGAAGTGCAAGAAATGACCGACATCACGTGGCTCAACTCCCCC
AGTCAGGTGGCGGTAGTGCCCTGGGGGTTGCGCAAAGTGCTGAACTGGCTGAAGTTCAAGTA
CGGAGACCTCCCCATGTACATAATATCCAACGGAATCGATGACGGGCTGCATGCTGAGGACG
ACCAGCTGAGGGTGTATTATATGCAGAATTACATAAACGAAGCTCTCAAAGCCCACATACTG
GATGGTATCAATCTTTGCGGATACTTTGCTTATTCGTTTAACGACCGCACAGCTCCGAGGTT
TGGCCTCTATCGTTATGCTGCAGATCAGTTTGAGCCCAAGGCATCCATGAAACATTACAGGA
AAATTATTGACAGCAATGGTTTCCCGGGCCCAGAAACTCTGGAAAGATTTTGTCCAGAAGAA
TTCACCGTGTGTACTGAGTGCAGTTTTTTTCACACCCGAAAGTCTCTCGAGTCTAGAGGGCC
CTACCCATACGATGTTCCAGATTACGCTTGATA

### Human KL2 with a Signal Peptide and a C-Terminal HA Tag, Amino Acid Sequence: (SEQ ID NO: 15)

The signal peptide is underlined. The first and last amino acids of the KL2 domain are underlined and marked in boldface. The HA tag is marked in boldface.

Sequence:

MPASAPPRRPRPPPPSLSLLLVLLGLGGRRLRARKSYCVDFAAIQPQIALLQEMHVTHFRFS
LDWALILPLGNQSQVNHTILQYYRCMASELVRVNITPVVALWQPMAPNQGLPRLLARQGAWE
NPYTALAFAEYARLCFQELGHHVKLWITMNEPYTRNMTYSAGHNLLKAHALAWHVYNEKFRH
AQNGKISIALQADWIEPACPFSQKDKEVAERVLEFDIGWLAEPIFGSGDYPWVMRDWLNQRN
NFLLPYFTEDEKKLIQGTFDFLALSHYTTILVDSEKEDPIKYNDYLEVQEMTDITWLNSPSQ
VAVVPWGLRKVLNWLKFKYGDLPMYIISNGIDDGLHAEDDQLRVYYMQNYINEALKAHILDG
INLCGYFAYSFNDRTAPREGLYRYAADQFEPKASMKHYRKIIDSNGFPGPETLERFCPEEFT
VCTECSFFHTRKSLESRGPYPYDVPDYA-

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-38. (canceled)

39. A recombinant polypeptide selected from the group consisting of:
a) a polypeptide comprising at least 280 contiguous amino acids of the human klotho polypeptide sequence as set forth in SEQ ID NO: 1, in which the amino acids at positions 341-1012 have been deleted, and at least the amino acids at positions 320-340 thereof are retained,
b) a polypeptide of 307-400 amino acids in length, comprising the amino acids at positions 34-340 of a native human klotho polypeptide precursor sequence, and
c) a polypeptide of at least 280 amino acids in length, having at least 90% sequence identity to a) or b), and exhibiting tumor-suppressive activity.

40. The polypeptide of claim 39, comprising, at its C-terminal end, the amino acid sequence LDFVLGWFAKPVFIDGDYPES (SEQ ID NO: 5), optionally followed by a heterologous sequence or moiety.

41. The recombinant polypeptide of claim 39, which is at least 300 amino acids in length.

42. The recombinant polypeptide of claim 39, consisting essentially of the amino acid sequence as set forth in SEQ ID NO: 3 or the amino acid sequence as set forth in SEQ ID NO: 3 excluding the signal peptide at positions 1-33 thereof.

43. The recombinant polypeptide of claim 39, encoded by the nucleic acid molecule of SEQ ID NO: 4.

44. The recombinant polypeptide of claim 39, comprising one or more heterologous sequences or moieties.

45. The recombinant polypeptide of claim 44, wherein the one or more heterologous sequences or moieties is linked at the C-terminal end or at the N-terminal end.

46. The recombinant polypeptide of claim 45, having an amino acid sequence as set forth in SEQ ID NO: 12 or 13.

47. The recombinant polypeptide of claim 44, wherein the one or more heterologous sequences or moieties is selected from the group consisting of a protein tag, a serum half-life elongating element and a therapeutic agent.

48. A nucleic acid molecule encoding the recombinant polypeptide of claim 39.

49. A recombinant construct, comprising the nucleic acid molecule of claim 48 that is operably linked to one or more transcription regulation sequences.

50. An expression vector comprising the recombinant construct of claim 49.

51. A host cell comprising the expression vector of claim 50.

52. A pharmaceutical composition comprising the polypeptide of claim 39 or a nucleic acid construct or vector encoding the same.

53. A method of treating cancer in a subject in need thereof, comprising administering to the subject, or expressing in cells of the subject, a recombinant polypeptide according to claim 39, thereby treating cancer in the subject.

54. The method of claim 53, wherein the subject is afflicted with a solid tumor.

55. The method of claim 54, wherein the tumor is selected from the group consisting of breast, pancreatic, colorectal, ovarian, cervical and lung tumors, glioblastoma and melanoma.

56. A method of inhibiting tumor progression in a subject in need thereof, comprising administering to the subject a therapeutically effective amount of a polypeptide according to claim 39, or a construct or vector encoding the same, thereby inhibiting tumor progression in the subject.

57. The method of claim 56, wherein the subject is afflicted with a solid tumor.

58. The method of claim 57, wherein the tumor is selected from the group consisting of breast, pancreatic, colorectal, ovarian, cervical and lung tumors, glioblastoma and melanoma.


SEQUENCE LISTING (15 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - DNA, 3039 bp, Homo sapiens
  source 1..3039: mol_type = genomic DNA
atgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatag

SEQ ID NO: 3 - protein, 340 aa, synthetic construct
  REGION 1..340: note = KL 1-340
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPES

SEQ ID NO: 4 - DNA, 1020 bp, synthetic construct
  misc_feature 1..1020: note = KL 1-340 polynucleotide
  source 1..1020: mol_type = other DNA
atgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagc

SEQ ID NO: 5 - protein, 21 aa, synthetic construct
  REGION 1..21: note = Positions 320-340 of human klotho
LDFVLGWFAKPVFIDGDYPES

SEQ ID NO: 6 - DNA, 1763 bp, synthetic construct
  misc_feature 1..1763: note = Human KL1 polynucleotide cloned in pcDNA3 with a C-terminal HA tag
  source 1..1763: mol_type = other DNA
ggatccgaattcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagctcgagtctagagggccctacccatacgatgttccagattacgcttgata

SEQ ID NO: 7 - protein, 582 aa, synthetic construct
  REGION 1..582: note = Human KL1 with a C-terminal HA tag
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKLESRGPYPYDVPDYA

SEQ ID NO: 8 - DNA, 1157 bp, synthetic construct
  misc_feature 1..1157: note = KL1-365 polynucleotide cloned in pcDNA3 with a C-terminal HA tag
  source 1..1157: mol_type = other DNA
ggatccgaattcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacctcgagtctagagggccctacccatacgatgttccagattacgcttgata

SEQ ID NO: 9 - DNA, 1076 bp, synthetic construct
  misc_feature 1..1076: note = KL1-340 polynucleotide cloned in pcDNA3 with a C-terminal HA tag
  source 1..1076: mol_type = other DNA
gaattcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcctcgagtctagagggccctacccatacgatgttccagattacgcttgata

SEQ ID NO: 10 - DNA, 1016 bp, synthetic construct
  misc_feature 1..1016: note = KL1-320 polynucleotide cloned in pcDNA3 with a C-terminal HA tag
  source 1..1016: mol_type = other DNA
gaattcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctgctcgagtctagagggccctacccatacgatgttccagattacgcttgata

SEQ ID NO: 11 - DNA, 1397 bp, synthetic construct
  misc_feature 1..1397: note = Human KL2 polynucleotide cloned in pcDNA3 with a C-terminal HA tag
  source 1..1397: mol_type = other DNA
gaattcatgcccgccagcgccccgccgcgccgcccgcggccgccgccgccgtcgctgtcgctgctgctggtgctgctgggcctgggcggccgccgcctgcgtgcgaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggccgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaacggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctctcgagtctagagggccctacccatacgatgttccagattacgcttgata

SEQ ID NO: 12 - protein, 380 aa, synthetic construct
  REGION 1..380: note = KL1-365 with a C-terminal HA tag
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADLESRGPYPYDVPDYA

SEQ ID NO: 13 - protein, 355 aa, synthetic construct
  REGION 1..355: note = KL1-340 with a C-terminal HA tag
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESLESRGPYPYDVPDYA

SEQ ID NO: 14 - protein, 335 aa, synthetic construct
  REGION 1..335: note = KL1-320 with a C-terminal HA tag
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLLESRGPYPYDVPDYA

SEQ ID NO: 15 - protein, 462 aa, synthetic construct
  REGION 1..462: note = Human KL2 with a signal peptide and a C-terminal HA tag
MPASAPPRRPRPPPPSLSLLLVLLGLGGRRLRARKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLESRGPYPYDVPDYA